(19) United States
(12) Patent Application Publication
(10) Pub. No.: US 2026/0345689 A1
(43) Pub. Date: Nov. 5, 2026

(54) COMPOSITION

(71) Applicants: Koji Tanabe, Palo Alto, CA (US); I Peace, Inc., Palo Alto, CA (US)

(72) Inventors: Koji TANABE, Palo Alto, CA (US); Kenta SUTO, Kyoto-shi, Kyoto (JP)

(73) Assignees: Koji Tanabe, Palo Alto, CA (US); I Peace, Inc., Palo Alto, CA (US)

(21) Appl. No.: 19/157,644

(22) PCT Filed: Feb. 19, 2024
(86) PCT No.: PCT/JP2024/005646

§ 371 Date: Dec. 16, 2025

(30) Foreign Application Priority Data
Feb. 20, 2023 (JP) 2023-024551

(51) Int. Cl. A61K 35/545; A61P 25/28

(52) U.S. Cl. CPC A61K 35/545; A61P 25/28

(57) ABSTRACT

A composition comprising at least one selected from the group consisting of a supernatant of a culture medium of stem cells, a supernatant of a culture medium used when reprogramming somatic cells, an exosome derived from stem cells, at least a part of stem cells, and an extract of stem cells; and a liposomal component. The composition may be a composition for preventing or ameliorating the formation of any of skin spots, wrinkles, or sagging. The liposomal component may comprise a phospholipid. The liposomal component may form a liposome. The supernatant of the culture medium of the stem cells may be a supernatant of a medium in which the stem cells have been maintained in an undifferentiated state. The stem cells may be reprogrammed stem cells. The stem cells may be derived from a subject to whom the composition is to be administered.

### TECHNICAL FIELD

The present invention relates to a composition.

### BACKGROUND ART

Embryonic stem cells (ES cells) are stem cells established from early embryos of humans or mice. ES cells have pluripotency, which is the ability to differentiate into all types of cells existing in a living organism. Currently, human ES cells are available for use in cell transplantation therapy for various diseases such as Parkinson's disease, juvenile diabetes, and leukemia. However, there are obstacles associated with the transplantation of ES cells. In particular, the transplantation of ES cells can induce immune rejection reactions similar to those that occur following unsuccessful organ transplantation. Furthermore, the use of ES cells, which are established by the destruction of human embryos, has been subject to considerable criticism and opposition from an ethical standpoint.

Under such circumstances, Professor Shinya Yamanaka of Kyoto University succeeded in establishing induced pluripotent stem cells (iPS cells) by introducing four genes—Oct3/4, Klf4, c-Myc, and Sox2—into somatic cells. As a result of this achievement, Professor Yamanaka was awarded the Nobel Prize in Physiology or Medicine in 2012 (see, for example, Patent Document 1). iPS cells are ideal pluripotent cells that are free from immune rejection and ethical issues. Therefore, iPS cells are expected to be utilized in cell transplantation therapy. On the other hand, there have been reports that the culture medium used for culturing iPS cells has been reused in pharmaceutical compositions (see, for example, Patent Document 2). However, upon examination by the present inventors, it was found that, in the culturing method described in Patent Document 2, the iPS cells undergo differentiation. Accordingly, it is believed that the culture medium actually reused is the medium used for culturing differentiated cells, rather than iPS cells themselves (see, for example, Patent Documents 3 and 4). Furthermore, in the field of cosmetics, liposomes have been used (see, for example, Patent Documents 5 to 7).

### PRIOR ART DOCUMENTS

### Patent Documents

Patent Document 1: Japanese U.S. Pat. No. 4,183,742
Patent Document 2: Japanese Unexamined Patent Publication No. 2016-128396
Patent Document 3: Japanese U.S. Pat. No. 6,647,545
Patent Document 4: Japanese U.S. Pat. No. 7,046,421
Patent Document 5: Japanese U.S. Pat. No. 6,242,422
Patent Document 6: Japanese U.S. Pat. No. 6,847,080
Patent Document 7: Japanese Unexamined Patent Publication No. 2014-214094

### SUMMARY OF THE INVENTION

### Problems to be Solved by the Invention

One of the objects of the present invention is to provide a composition that effectively utilizes components of stem cells such as iPS cells and culture fluids of stem cells.

### Means for Solving the Problems

A composition according to an embodiment comprises at least one selected from the group consisting of a supernatant of a culture medium of stem cells, a supernatant of a culture medium used when reprogramming somatic cells, a secretion derived from stem cells or somatic cells into which reprogramming factors have been introduced, an exosome derived from stem cells, at least a portion of stem cells; and an extract of stem cells; and a liposomal component.

The composition may be a pharmaceutical composition or a cosmetic composition.

The composition according to the above embodiment may be a composition for improving the softness of the skin.

The composition according to the above embodiment may be a composition for improving the elasticity of the skin.

The composition according to the above embodiment may be a composition for increasing the moisture content of the stratum corneum.

The composition according to the above embodiment may be a composition for improving the firmness of the skin.

The composition according to the above embodiment may be a composition for reducing pigmentation spots.

The composition according to the above embodiment may be a composition for preventing and ameliorating the formation of any of pigmentation spots, wrinkles, or sagging of the skin.

The composition according to the above embodiment may be a composition for promoting collagen production.

The composition according to the above embodiment may be a composition for promoting hyaluronic acid production.

The composition according to the above embodiment may be a composition for treating wounds.

The composition according to the above embodiment may be a composition for promoting epidermal cell proliferation.

The composition according to the above embodiment may be a composition for promoting hair growth or hair nourishment.

The composition according to the above embodiment may be a composition for activating hair papilla cells.

The composition according to the above embodiment may be a composition for promoting the production of fibroblast growth factor (FGF) family.

The composition according to the above embodiment may be a composition for promoting the production of vascular endothelial growth factor (VEGF).

The composition according to the above embodiment may be a composition for protecting cells.

The composition according to the above embodiment may be a composition for protecting cells from stress.

The composition according to the above embodiment may be a composition for improving the survival rate of stressed cells.

The composition according to the above embodiment may be a composition for protecting biological substances.

In the composition according to the above embodiment, the liposomal component may contain a lipid. The lipid may be a phospholipid.

In the composition according to the above embodiment, the liposomal component may form a liposome.

In the composition according to the above embodiment, the supernatant of the culture medium of the stem cells may be a supernatant of a medium in which the stem cells have been maintained in an undifferentiated state.

In the composition according to the above embodiment, the supernatant of the culture medium of the stem cells may be a supernatant of the culture medium obtained when the stem cells have been subjected to two-dimensional culture.

In the composition according to the above embodiment, the supernatant of the culture medium of the stem cells may be a supernatant of the culture medium obtained when the stem cells have been subjected to three-dimensional culture.

In the composition according to the above embodiment, the stem cells may be pluripotent stem cells.

In the composition according to the above embodiment, the stem cells may be reprogrammed stem cells.

In the composition according to the above embodiment, the stem cells may be derived from the subject to whom the composition is to be administered.

The composition according to the above embodiment may be an agent for lipolysis, an anti-obesity agent, an agent for ameliorating obesity, an agent for preventing obesity, a lipolysis-promoting agent, a diet agent, a therapeutic agent for diabetes, a prophylactic agent for diabetes, a prostacyclin production-promoting agent, a cyclic adenosine monophosphate production-promoting agent, an anti-platelet aggregation agent, a vasodilator, a therapeutic agent for hypertension, a prophylactic agent for hypertension, a therapeutic agent for arteriosclerosis, a prophylactic agent for arteriosclerosis, a vascular endothelial cell proliferation inhibitor, a hyaluronic acid production-promoting agent, a hyaluronic acid production-promoting agent for chondrocytes, a proliferation-promoting agent for chondrocytes, a therapeutic agent for cartilage tissue wounds, a prophylactic agent for cartilage tissue wounds, a therapeutic agent for joint diseases, a prophylactic agent for joint diseases, a therapeutic agent for joint pain, a prophylactic agent for joint pain, an agent for improving the survival rate of chondrocytes, a therapeutic agent for lifestyle-related diseases, a prophylactic agent for lifestyle-related diseases, a therapeutic agent for adult diseases, a prophylactic agent for adult diseases, an amyloid β inhibitor, a therapeutic agent for neurological diseases, a prophylactic agent for neurological diseases, a therapeutic agent for dementia, a prophylactic agent for dementia, an inhibitor of neuronal cell death, a therapeutic agent for reduction in neuronal cell number, a prophylactic agent for reduction in neuronal cell number, a collagen production-promoting agent, an agent for preventing the formation of any of pigmentation spots, wrinkles, or sagging of the skin, an agent for ameliorating any of pigmentation spots, wrinkles, or sagging of the skin, a fibroblast growth factor (FGF) family production-promoting agent, a vascular endothelial growth factor (VEGF) production-promoting agent, a hair growth agent, a hair nourishing agent, a fibroblast proliferation-promoting agent, or a wound-healing agent.

The composition according to the above embodiment may be an injection preparation or an infusion preparation.

According to an embodiment, there is provided the use of a composition comprising at least one selected from the group consisting of a supernatant of a culture medium of stem cells, a supernatant of a culture medium used when reprogramming somatic cells, a secretion derived from stem cells or from somatic cells into which reprogramming factors have been introduced, an exosome derived from stem cells, at least a portion of stem cells, and an extract of stem cells; and a liposomal component, in the manufacture of any of the following: an agent for lipolysis, an anti-obesity agent, an agent for ameliorating obesity, an agent for preventing obesity, a lipolysis-promoting agent, a diet agent, a therapeutic agent for diabetes, a prophylactic agent for diabetes, a prostacyclin production-promoting agent, a cyclic adenosine monophosphate production-promoting agent, an anti-platelet aggregation agent, a vasodilator, a therapeutic agent for hypertension, a prophylactic agent for hypertension, a therapeutic agent for arteriosclerosis, a prophylactic agent for arteriosclerosis, a vascular endothelial cell proliferation inhibitor, a hyaluronic acid production-promoting agent, a hyaluronic acid production-promoting agent for chondrocytes, a proliferation-promoting agent for chondrocytes, a therapeutic agent for cartilage tissue wounds, a prophylactic agent for cartilage tissue wounds, a therapeutic agent for joint diseases, a prophylactic agent for joint diseases, a therapeutic agent for joint pain, a prophylactic agent for joint pain, an agent for improving the survival rate of chondrocytes, a therapeutic agent for lifestyle-related diseases, a prophylactic agent for lifestyle-related diseases, a therapeutic agent for adult diseases, a prophylactic agent for adult diseases, an amyloid β inhibitor, a therapeutic agent for neurological diseases, a prophylactic agent for neurological diseases, a therapeutic agent for dementia, a prophylactic agent for dementia, an inhibitor of neuronal cell death, a therapeutic agent for reduction in neuronal cell number, a prophylactic agent for reduction in neuronal cell number, a collagen production-promoting agent, an agent for preventing the formation of any of pigmentation spots, wrinkles, or sagging of the skin, an agent for ameliorating any of pigmentation spots, wrinkles, or sagging of the skin, a fibroblast growth factor (FGF) family production-promoting agent, a vascular endothelial growth factor (VEGF) production-promoting agent, a hair growth agent, a hair nourishing agent, a fibroblast proliferation-promoting agent, or a wound-healing agent.

According to an embodiment, there is provided the use of a composition comprising at least one selected from the group consisting of a supernatant of a culture medium of stem cells, a supernatant of a culture medium used when reprogramming somatic cells, a secretion derived from stem cells or from somatic cells into which reprogramming factors have been introduced, an exosome derived from stem cells, at least a portion of stem cells; and an extract of stem cells; and a liposomal component, for lipolysis, anti-obesity, amelioration of obesity, prevention of obesity, promotion of lipolysis, dieting, treatment of diabetes, prevention of diabetes, promotion of prostacyclin production, promotion of cyclic adenosine monophosphate production, inhibition of platelet aggregation, vasodilation, treatment of hypertension, prevention of hypertension, treatment of arteriosclerosis, prevention of arteriosclerosis, inhibition of vascular endothelial cell proliferation, promotion of hyaluronic acid production, promotion of hyaluronic acid production by chondrocytes, promotion of chondrocyte proliferation, treatment of cartilage tissue wounds, prevention of cartilage tissue wounds, treatment of joint diseases, prevention of joint diseases, treatment of joint pain, prevention of joint pain, improvement of chondrocyte survival rate, treatment of lifestyle-related diseases, prevention of lifestyle-related diseases, treatment of adult diseases, prevention of adult diseases, inhibition of amyloid, treatment of neurological diseases, prevention of neurological diseases, treatment of dementia, prevention of dementia, inhibition of neuronal cell death, treatment of neuronal cell loss, prevention of neuronal cell loss, promotion of collagen production, prevention of formation of any of pigmentation spots, wrinkles, or sagging of the skin, amelioration of any of pigmentation spots, wrinkles, or sagging of the skin, promotion of fibroblast growth factor (FGF) family production, promotion of vascular endothelial growth factor (VEGF) production, hair growth, hair nourishment, promotion of fibroblast proliferation, or wound healing.

According to an embodiment, there is provided a method for lipolysis, an anti-obesity method, a method for ameliorating obesity, a method for preventing obesity, a method for promoting lipolysis, a dieting method, a method for treating diabetes, a method for preventing diabetes, a method for promoting prostacyclin production, a method for promoting cyclic adenosine monophosphate production, a method for inhibiting platelet aggregation, a vasodilation method, a method for treating hypertension, a method for preventing hypertension, a method for treating arteriosclerosis, a method for preventing arteriosclerosis, a method for inhibiting vascular endothelial cell proliferation, a method for promoting hyaluronic acid production, a method for promoting hyaluronic acid production in chondrocytes, a method for promoting chondrocyte proliferation, a method for treating cartilage tissue wounds, a method for preventing cartilage tissue wounds, a method for treating joint diseases, a method for preventing joint diseases, a method for treating joint pain, a method for preventing joint pain, a method for improving chondrocyte survival rate, a method for treating lifestyle-related diseases, a method for preventing lifestyle-related diseases, a method for treating adult diseases, a method for preventing adult diseases, a method for inhibiting amyloid β, a method for treating neurological diseases, a method for preventing neurological diseases, a method for treating dementia, a method for preventing dementia, a method for inhibiting neuronal cell death, a method for treating neuronal cell loss, a method for preventing neuronal cell loss, a method for promoting collagen production, a method for preventing the formation of any of pigmentation spots, wrinkles, or sagging of the skin, a method for ameliorating any of pigmentation spots, wrinkles, or sagging of the skin, a method for promoting fibroblast growth factor (FGF) family production, a method for promoting vascular endothelial growth factor (VEGF) production, a method for promoting hair growth, a method for promoting hair nourishment, a method for promoting fibroblast proliferation, or a wound healing method, the method comprising administering to a subject a composition comprising at least one selected from the group consisting of a supernatant of a culture medium of stem cells, a supernatant of a culture medium used when reprogramming somatic cells, a secretion derived from stem cells or from somatic cells into which reprogramming factors have been introduced, an exosome derived from stem cells, at least a portion of stem cells, and an extract of stem cells; and a liposomal component.

The subject is a human, a non-human animal, or cells thereof.

A cosmetic method according to an embodiment comprises administering to a subject a cosmetic comprising at least one selected from the group consisting of a supernatant of a culture medium of stem cells, a supernatant of a culture medium used when reprogramming somatic cells, a secretion derived from stem cells or from somatic cells into which reprogramming factors have been introduced, an exosome derived from stem cells, at least a portion of stem cells, and an extract of stem cells; and a liposomal component.

The administration may be performed by applying the cosmetic to the skin.

The cosmetic method according to the above embodiment may be a cosmetic method for improving the softness of the skin.

The cosmetic method according to the above embodiment may be a cosmetic method for improving the elasticity of the skin.

The cosmetic method according to the above embodiment may be a cosmetic method for increasing the moisture content of the stratum corneum.

The cosmetic method according to the above embodiment may be a cosmetic method for improving the firmness of the skin.

The cosmetic method according to the above embodiment may be a cosmetic method for reducing pigmentation spots.

The cosmetic method according to the above embodiment may be a cosmetic method for preventing and ameliorating the formation of any of pigmentation spots, wrinkles, or sagging of the skin.

The cosmetic method according to the above embodiment may be a cosmetic method for promoting collagen production.

The cosmetic method according to the above embodiment may be a cosmetic method for promoting hyaluronic acid production.

The cosmetic method according to the above embodiment may be a cosmetic method for making wounds less noticeable.

The cosmetic method according to the above embodiment may be a cosmetic method for promoting epidermal cell proliferation.

The cosmetic method according to the above embodiment may be a cosmetic method for promoting hair growth or hair nourishment.

The cosmetic method according to the above embodiment may be a cosmetic method for activating hair papilla cells.

The cosmetic method according to the above embodiment may be a cosmetic method for promoting fibroblast growth factor (FGF) family production.

The cosmetic method according to the above embodiment may be a cosmetic method for promoting vascular endothelial growth factor (VEGF) production.

The cosmetic method according to the above embodiment may be a cosmetic method for protecting cells.

The cosmetic method according to the above embodiment may be a cosmetic method for protecting cells from stress.

The cosmetic method according to the above embodiment may be a cosmetic method for improving the survival rate of stressed cells.

The cosmetic method according to the above embodiment may be a cosmetic method for protecting biological substances.

In the cosmetic method according to the above embodiment, the liposomal component of the cosmetic may contain a lipid. The lipid may be a phospholipid.

In the cosmetic method according to the above embodiment, the liposomal component of the cosmetic may form a liposome.

In the cosmetic method according to the above embodiment, the supernatant of the culture medium of stem cells contained in the cosmetic may be a supernatant of a medium in which the stem cells have been maintained in an undifferentiated state.

In the cosmetic method according to the above embodiment, the supernatant of the culture medium of stem cells contained in the cosmetic may be a supernatant of the culture medium obtained when the stem cells have been subjected to two-dimensional culture.

In the cosmetic method according to the above embodiment, the supernatant of the culture medium of stem cells contained in the cosmetic may be a supernatant of the culture medium obtained when the stem cells have been subjected to three-dimensional culture.

In the cosmetic method according to the above embodiment, the stem cells may be pluripotent stem cells.

In the cosmetic method according to the above embodiment, the stem cells may be reprogrammed stem cells.

In the cosmetic method according to the above embodiment, the stem cells may be derived from the subject to whom the cosmetic is to be administered.

A method for producing a composition according to an embodiment comprises mixing at least one selected from the group consisting of a supernatant of a culture medium of stem cells, a supernatant of a culture medium used when reprogramming somatic cells, a secretion derived from stem cells or from somatic cells into which reprogramming factors have been introduced, an exosome derived from stem cells, at least a portion of stem cells, and an extract of stem cells; and a liposomal component, and forming a liposome encapsulating at least one selected from the group consisting of the supernatant of the culture medium of the stem cells, the supernatant of the culture medium used when reprogramming the somatic cells, the exosome derived from the stem cells, at least a portion of the stem cells, and the extract of the stem cells.

The composition produced by the method for producing a composition according to the embodiment may be the above-described composition. The composition may be a pharmaceutical composition or a cosmetic composition.

A method for producing a composition according to an embodiment comprises mixing at least one selected from the group consisting of a supernatant of a culture medium of stem cells, a supernatant of a culture medium used when reprogramming somatic cells, a secretion derived from stem cells or from somatic cells into which reprogramming factors have been introduced, an exosome derived from stem cells, at least a portion of stem cells, and an extract of stem cells; and a liposome. The composition produced by the method for producing a composition according to the embodiment may be the above-described composition. The composition may be a pharmaceutical composition or a cosmetic composition.

According to an embodiment, there is provided a pharmaceutical or cosmetic product comprising a secretion derived from stem cells or from somatic cells into which reprogramming factors have been introduced.

The pharmaceutical or cosmetic product described above may be any of the following: a lipolysis agent, an anti-obesity agent, an agent for ameliorating obesity, an agent for preventing obesity, a lipolysis-promoting agent, a diet agent, a therapeutic agent for diabetes, a prophylactic agent for diabetes, a prostacyclin production-promoting agent, a cyclic adenosine monophosphate production-promoting agent, an anti-platelet aggregation agent, a vasodilator, a therapeutic agent for hypertension, a prophylactic agent for hypertension, a therapeutic agent for arteriosclerosis, a prophylactic agent for arteriosclerosis, a vascular endothelial cell proliferation inhibitor, a hyaluronic acid production-promoting agent, a hyaluronic acid production-promoting agent for chondrocytes, a proliferation-promoting agent for chondrocytes, a therapeutic agent for cartilage tissue wounds, a prophylactic agent for cartilage tissue wounds, a therapeutic agent for joint diseases, a prophylactic agent for joint diseases, a therapeutic agent for joint pain, a prophylactic agent for joint pain, an agent for improving chondrocyte survival rate, a therapeutic agent for lifestyle-related diseases, a prophylactic agent for lifestyle-related diseases, a therapeutic agent for adult diseases, a prophylactic agent for adult diseases, an amyloid β inhibitor, a therapeutic agent for neurological diseases, a prophylactic agent for neurological diseases, a therapeutic agent for dementia, a prophylactic agent for dementia, an inhibitor of neuronal cell death, a therapeutic agent for reduction in neuronal cell number, a prophylactic agent for reduction in neuronal cell number, a collagen production-promoting agent, an agent for preventing the formation of any of pigmentation spots, wrinkles, or sagging of the skin, an agent for ameliorating any of pigmentation spots, wrinkles, or sagging of the skin, a fibroblast growth factor (FGF) family production-promoting agent, a vascular endothelial growth factor (VEGF) production-promoting agent, a hair growth agent, a hair nourishing agent, a fibroblast proliferation-promoting agent, or a wound healing agent.

In the pharmaceutical or cosmetic product described above, the stem cells may be pluripotent stem cells, totipotent stem cells, or multipotent stem cells.

In the pharmaceutical or cosmetic product described above, the stem cells may be reprogrammed stem cells.

In the pharmaceutical or cosmetic product described above, the stem cells may be derived from the subject to whom the pharmaceutical or cosmetic product is to be administered.

In the pharmaceutical or cosmetic product described above, the secretion may be extracellular vesicles.

In the pharmaceutical or cosmetic product described above, the secretion may be an exosome.

In the pharmaceutical or cosmetic product described above, the secretion may be a protein.

In the pharmaceutical or cosmetic product described above, the secretion may be an isolated secretion.

In the pharmaceutical or cosmetic product described above, the secretion may be a concentrated secretion.

In the pharmaceutical or cosmetic product described above, the product may be an injection preparation or an infusion preparation.

According to an embodiment, there is provided the use of a secretion derived from stem cells or from somatic cells into which reprogramming factors have been introduced in the manufacture of a pharmaceutical or cosmetic product.

In the use of the secretion derived from stem cells or from somatic cells into which reprogramming factors have been introduced, the pharmaceutical or cosmetic product may be any of the following: a lipolysis agent, an anti-obesity agent, an agent for ameliorating obesity, an agent for preventing obesity, a lipolysis-promoting agent, a diet agent, a therapeutic agent for diabetes, a prophylactic agent for diabetes, a prostacyclin production-promoting agent, a cyclic adenosine monophosphate production-promoting agent, an anti-platelet aggregation agent, a vasodilator, a therapeutic agent for hypertension, a prophylactic agent for hypertension, a therapeutic agent for arteriosclerosis, a prophylactic agent for arteriosclerosis, a vascular endothelial cell proliferation inhibitor, a hyaluronic acid production-promoting agent, a hyaluronic acid production-promoting agent for chondrocytes, a proliferation-promoting agent for chondrocytes, a therapeutic agent for cartilage tissue wounds, a prophylactic agent for cartilage tissue wounds, a therapeutic agent for joint diseases, a prophylactic agent for joint diseases, a therapeutic agent for joint pain, a prophylactic agent for joint pain, an agent for improving chondrocyte survival rate, a therapeutic agent for lifestyle-related diseases, a prophylactic agent for lifestyle-related diseases, a therapeutic agent for adult diseases, a prophylactic agent for adult diseases, an amyloid β inhibitor, a therapeutic agent for neurological diseases, a prophylactic agent for neurological diseases, a therapeutic agent for dementia, a prophylactic agent for dementia, an inhibitor of neuronal cell death, a therapeutic agent for reduction in neuronal cell number, a prophylactic agent for reduction in neuronal cell number, a collagen production-promoting agent, an agent for preventing the formation of any of pigmentation spots, wrinkles, or sagging of the skin, an agent for ameliorating any of pigmentation spots, wrinkles, or sagging of the skin, a fibroblast growth factor (FGF) family production-promoting agent, a vascular endothelial growth factor (VEGF) production-promoting agent, a hair growth agent, a hair nourishing agent, a fibroblast proliferation-promoting agent, or a wound healing agent.

According to an embodiment, there is provided the use of a secretion derived from stem cells or from somatic cells into which reprogramming factors have been introduced for lipolysis, anti-obesity, amelioration of obesity, prevention of obesity, promotion of lipolysis, dieting, treatment of diabetes, prevention of diabetes, promotion of prostacyclin production, promotion of cyclic adenosine monophosphate production, inhibition of platelet aggregation, vasodilation, treatment of hypertension, prevention of hypertension, treatment of arteriosclerosis, prevention of arteriosclerosis, inhibition of vascular endothelial cell proliferation, promotion of hyaluronic acid production, promotion of hyaluronic acid production in chondrocytes, promotion of chondrocyte proliferation, treatment of cartilage tissue wounds, prevention of cartilage tissue wounds, treatment of joint diseases, prevention of joint diseases, treatment of joint pain, prevention of joint pain, improvement of chondrocyte survival rate, treatment of lifestyle-related diseases, prevention of lifestyle-related diseases, treatment of adult diseases, prevention of adult diseases, inhibition of amyloid β, treatment of neurological diseases, prevention of neurological diseases, treatment of dementia, prevention of dementia, inhibition of neuronal cell death, treatment of neuronal cell loss, prevention of neuronal cell loss, promotion of collagen production, prevention of the formation of any of pigmentation spots, wrinkles, or sagging of the skin, amelioration of any of pigmentation spots, wrinkles, or sagging of the skin, promotion of fibroblast growth factor (FGF) family production, promotion of vascular endothelial growth factor (VEGF) production, hair growth, hair nourishment, promotion of fibroblast proliferation, or wound healing.

According to an embodiment, there is provided a method for lipolysis, an anti-obesity method, a method for ameliorating obesity, a method for preventing obesity, a method for promoting lipolysis, a dieting method, a method for treating diabetes, a method for preventing diabetes, a method for promoting prostacyclin production, a method for promoting cyclic adenosine monophosphate production, a method for inhibiting platelet aggregation, a vasodilation method, a method for treating hypertension, a method for preventing hypertension, a method for treating arteriosclerosis, a method for preventing arteriosclerosis, a method for inhibiting vascular endothelial cell proliferation, a method for promoting hyaluronic acid production, a method for promoting hyaluronic acid production in chondrocytes, a method for promoting chondrocyte proliferation, a method for treating cartilage tissue wounds, a method for preventing cartilage tissue wounds, a method for treating joint diseases, a method for preventing joint diseases, a method for treating joint pain, a method for preventing joint pain, a method for improving chondrocyte survival rate, a method for treating lifestyle-related diseases, a method for preventing lifestyle-related diseases, a method for treating adult diseases, a method for preventing adult diseases, a method for inhibiting amyloid β, a method for treating neurological diseases, a method for preventing neurological diseases, a method for treating dementia, a method for preventing dementia, a method for inhibiting neuronal cell death, a method for treating reduction in neuronal cell number, a method for preventing reduction in neuronal cell number, a method for promoting collagen production, a method for preventing the formation of any of pigmentation spots, wrinkles, or sagging of the skin, a method for ameliorating any of pigmentation spots, wrinkles, or sagging of the skin, a method for promoting fibroblast growth factor (FGF) family production, a method for promoting vascular endothelial growth factor (VEGF) production, a hair growth method, a hair nourishment method, a method for promoting fibroblast proliferation, or a wound healing method, the method comprising administering to a subject a secretion derived from stem cells or from somatic cells into which reprogramming factors have been introduced.

The subject is a human, a non-human animal, or cells thereof.

According to an embodiment, there is provided a method for producing a pharmaceutical or cosmetic product, the method comprising collecting a secretion derived from stem cells or from somatic cells into which reprogramming factors have been introduced.

In the method for producing the pharmaceutical or cosmetic product described above, the pharmaceutical or cosmetic product may be any of the following: a lipolysis agent, an anti-obesity agent, an agent for ameliorating obesity, an agent for preventing obesity, a lipolysis-promoting agent, a diet agent, a therapeutic agent for diabetes, a prophylactic agent for diabetes, a prostacyclin production-promoting agent, a cyclic adenosine monophosphate production-promoting agent, an anti-platelet aggregation agent, a vasodilator, a therapeutic agent for hypertension, a prophylactic agent for hypertension, a therapeutic agent for arteriosclerosis, a prophylactic agent for arteriosclerosis, a vascular endothelial cell proliferation inhibitor, a hyaluronic acid production-promoting agent, a hyaluronic acid production-promoting agent for chondrocytes, a proliferation-promoting agent for chondrocytes, a therapeutic agent for cartilage tissue wounds, a prophylactic agent for cartilage tissue wounds, a therapeutic agent for joint diseases, a prophylactic agent for joint diseases, a therapeutic agent for joint pain, a prophylactic agent for joint pain, an agent for improving chondrocyte survival rate, a therapeutic agent for lifestyle-related diseases, a prophylactic agent for lifestyle-related diseases, a therapeutic agent for adult diseases, a prophylactic agent for adult diseases, an amyloid β inhibitor, a therapeutic agent for neurological diseases, a prophylactic agent for neurological diseases, a therapeutic agent for dementia, a prophylactic agent for dementia, an inhibitor of neuronal cell death, a therapeutic agent for reduction in neuronal cell number, a prophylactic agent for reduction in neuronal cell number, a collagen production-promoting agent, an agent for preventing the formation of any of pigmentation spots, wrinkles, or sagging of the skin, an agent for ameliorating any of pigmentation spots, wrinkles, or sagging of the skin, a fibroblast growth factor (FGF) family production-promoting agent, a vascular endothelial growth factor (VEGF) production-promoting agent, a hair growth agent, a hair nourishing agent, a fibroblast proliferation-promoting agent, or a wound healing agent.

The method for producing the pharmaceutical or cosmetic product described above may further include isolating the secretion.

The method for producing the pharmaceutical or cosmetic product described above may further include concentrating the secretion.

In the method for producing the pharmaceutical or cosmetic product described above, the step of collecting the secretion derived from stem cells or from somatic cells into which reprogramming factors have been introduced may include collecting the secretion from the supernatant of the culture medium of the stem cells or the somatic cells into which reprogramming factors have been introduced.

In the method for producing the pharmaceutical or cosmetic product described above, the step of collecting the secretion derived from stem cells or from somatic cells into which reprogramming factors have been introduced may include collecting the secretion from at least a portion of the stem cells or the somatic cells into which reprogramming factors have been introduced.

In the method for producing the pharmaceutical or cosmetic product described above, the step of collecting the secretion derived from stem cells or from somatic cells into which reprogramming factors have been introduced may include collecting the secretion from an extract of the stem cells or the somatic cells into which reprogramming factors have been introduced.

In the method for producing the pharmaceutical or cosmetic product described above, the stem cells may be pluripotent stem cells, totipotent stem cells, or multipotent stem cells.

In the method for producing the pharmaceutical or cosmetic product described above, the stem cells may be reprogrammed stem cells.

In the method for producing the pharmaceutical or cosmetic product described above, the stem cells may be derived from the subject to whom the pharmaceutical or cosmetic product is to be administered.

In the method for producing the pharmaceutical or cosmetic product described above, the secretion may be extracellular vesicles.

In the method for producing the pharmaceutical or cosmetic product described above, the secretion may be an exosome.

In the method for producing the pharmaceutical or cosmetic product described above, the secretion may be a protein.

In the method for producing the pharmaceutical or cosmetic product described above, the pharmaceutical or cosmetic product may be an injection preparation or an infusion preparation.

### Advantages of the Invention

According to the present invention, it is possible to provide a composition that effectively utilizes components of stem cells and culture fluids of stem cells.

### BRIEF DESCRIPTION OF THE DRAWINGS

FIG. 1 A graph illustrating the method for measuring skin properties according to Example 3.

FIG. 2 A table showing changes in skin softness according to Example 3.

FIG. 3 A graph showing changes in skin softness according to Example 3.

FIG. 4 A table showing changes in skin elasticity according to Example 3.

FIG. 5 A graph showing changes in skin elasticity according to Example 3.

FIG. 6 A table showing changes in moisture content of the stratum corneum according to Example 3.

FIG. 7 A graph showing changes in moisture content of the stratum corneum according to Example 3.

FIG. 8 A table and graph showing changes in skin elasticity according to Example 4.

FIG. 9 A table and graph showing changes in skin firmness according to Example 4.

FIG. 10 A table and graph showing changes in moisture content of the stratum corneum according to Example 4.

FIG. 11 A table and graph showing changes in skin pigmentation spots according to Example 4.

FIG. 12 A table showing evaluation results according to Example 5.

FIG. 13 A table showing evaluation results according to Example 5.

FIG. 14 A graph showing results of fibroblast proliferation test according to Reference Example 4.

FIG. 15 A graph showing results of fibroblast proliferation test according to Reference Example 4.

FIG. 16 A graph showing results of collagen production test by fibroblasts according to Reference Example 5.

FIG. 17 A graph showing results of collagen production test by fibroblasts according to Reference Example 5.

FIG. 18 A graph showing results of hyaluronic acid production test by fibroblasts according to Reference Example 5.

FIG. 19 A photograph showing results of epidermal cell migration test according to Reference Example 6.

FIG. 20 A graph showing results of epidermal cell migration test according to Reference Example 6.

FIG. 21 A graph showing results of hair papilla cell proliferation test according to Reference Example 7.

FIG. 22 A graph showing results of FGF-7 production test by hair papilla cells according to Reference Example 8.

FIG. 23 A graph showing results of VEGF production test by hair papilla cells according to Reference Example 8.

FIG. 24 A table showing measurement results of free glycerol when the test substance according to Example 7 was added to the culture medium of adipocytes.

FIG. 25 A graph showing measurement results of free glycerol when the test substance according to Example 7 was added to the culture medium of adipocytes.

FIG. 26 A table showing measurement results of 6-keto prostaglandin Fla when the test substance according to Example 8 was added to the culture medium of normal human umbilical vein endothelial cells.

FIG. 27 A graph showing the amount of 6-keto prostaglandin Fla when the test substance according to Example 8 was added to the culture medium of normal human umbilical vein endothelial cells.

FIG. 28 A table showing measurement results of hyaluronic acid production when the test substance according to Example 9 was added to the culture medium of human chondrocytes and cultured.

FIG. 29 A graph showing hyaluronic acid production when the test substance according to Example 9 was added to the culture medium of human chondrocytes and cultured.

FIG. 30 A table showing measurement results of hyaluronic acid production when the test substance according to Example 10 was added to the culture medium of human fibroblasts and cultured.

FIG. 31 A graph showing hyaluronic acid production when the test substance according to Example 10 was added to the culture medium of human fibroblasts and cultured.

FIG. 32 A table showing measurement results of collagen production when the test substance according to Example 11 was added to the culture medium of human fibroblasts and cultured.

FIG. 33 A graph showing collagen production when the test substance according to Example 11 was added to the culture medium of human fibroblasts and cultured.

FIG. 34 A table showing measurement results of FGF-7 production when the test substance according to Example 12 was added to the culture medium of human hair papilla cells and cultured.

FIG. 35 A graph showing FGF-7 production when the test substance according to Example 12 was added to the culture medium of human hair papilla cells and cultured.

FIG. 36 A table showing measurement results of VEGF production when the test substance according to Example 13 was added to the culture medium of human hair papilla cells and cultured.

FIG. 37 A graph showing VEGF production when the test substance according to Example 13 was added to the culture medium of human hair papilla cells and cultured.

FIG. 38 A table showing measurement results of fibroblast cell number when the test substance according to Example 14 was added to the culture medium of human fibroblasts and cultured.

FIG. 39 A graph showing fibroblast cell number when the test substance according to Example 14 was added to the culture medium of human fibroblasts and cultured.

FIG. 40 A table showing measurement results of cerebral neuron cell number when the test substance according to Example 16 was added to the culture medium of cerebral neurons and cultured.

FIG. 41 A graph showing cerebral neuron cell number when the test substance according to Example 16 was added to the culture medium of cerebral neurons and cultured.

### DESCRIPTION OF EMBODIMENTS

Hereinafter, embodiments of the present invention will be described in detail. It should be noted that the embodiments described below illustrate apparatuses and methods for embodying the technical idea of the present invention, and the technical idea of the invention is not limited to the combinations of the components described below. Various modifications can be made to the technical idea of the invention within the scope of the claims.

A composition according to the embodiment contains at least one selected from the group consisting of a supernatant of a culture medium of stem cells, a supernatant of a culture medium used when reprogramming somatic cells, a secretion derived from stem cells or from somatic cells into which reprogramming factors have been introduced, an exosome derived from stem cells, at least a portion of stem cells, and an extract of stem cells; and a liposomal component. The composition may be a pharmaceutical composition or a cosmetic composition. The culture medium of the stem cells may be a culture medium in which the stem cells have been maintained in an undifferentiated state. The stem cells may be, for example, pluripotent stem cells such as induced pluripotent stem (iPS) cells or embryonic stem(ES) cells. The stem cells may also be reprogrammed cells, for example. Furthermore, the stem cells may include somatic stem cells and precursor cells induced from pluripotent stem cells. Examples of somatic stem cells include mesenchymal stem cells, hematopoietic stem cells, and neural stem cells. The stem cells may be subjected to two-dimensional culture or three-dimensional culture. The stem cells may consist solely of iPS cells and may not include other types of stem cells.

As the culture medium for stem cells, for example, human ES/iPS culture media such as TeSR2 (STEMCELL Technologies) may be used. However, the stem cell culture medium is not limited thereto, and various other stem cell culture media may also be used.

Alternatively, as the stem cell culture medium, a Dulbecco's Modified Eagle Medium/Ham's F-12 (DMEM/F-12) supplemented with serum substitute, L-glutamine, non-essential amino acid solution, 2-mercaptoethanol, and penicillin/streptomycin may be used. The stem cell culture medium may also contain growth factors such as basic fibroblast growth factor (bFGF).

The two-dimensional culture includes adherent culture. The three-dimensional culture includes suspension culture in which cells are able to float in the culture medium. In the suspension culture, the culture medium may be stirred or may remain unstirred. Additionally, the three-dimensional culture includes a culture in which cells are immobilized within a three-dimensional matrix and do not float. When the stem cells are cultured in three dimensions, the culture medium may be a liquid medium or a gel medium. The gel medium may be prepared, for example, by adding gellan gum, such as deacylated gellan gum, to the stem cell culture medium so that the final concentration of the gellan gum is from 0.5% by weight to 0.001% by weight, from 0.1% by weight to 0.005% by weight, or from 0.05% by weight to 0.01% by weight. It should be noted that in the present disclosure, “gellan gum” includes deacylated gellan gum.

The culture medium may contain at least one high-molecular-weight compound selected from the group consisting of hyaluronic acid, rhamsan gum, diutan gum, xanthan gum, carrageenan, fucoidan, pectin, pectic acid, pectinic acid, heparan sulfate, heparin, heparitin sulfate, keratan sulfate, chondroitin sulfate, dermatan sulfate, rhamnan sulfate, and salts thereof. In addition, the culture medium may contain methylcellulose, as well as lipids such as lysophosphatidic acid and sphingosine-1-phosphate. By including these substances, aggregation of cells can be suppressed. Further, the culture medium may contain natural or artificial extracellular matrices. Examples of extracellular matrices include Matrigel, laminin, vitronectin, and fibronectin. Moreover, the culture medium may contain polyethylene glycol (PEG).

The culture medium may contain at least one temperature-sensitive gel selected from the group consisting of poly(glycerol monomethacrylate) (PGMA), poly(2-hydroxypropyl methacrylate) (PHPMA), poly(N-isopropylacrylamide) (PNIPAM), amine-terminated, carboxylic acid terminated, maleimide terminated, N-hydroxysuccinimide (NHS) ester terminated, triethoxysilane terminated, poly(N-isopropylacrylamide-co-acrylamide), poly(N-isopropylacrylamide-co-acrylic acid), poly(N-isopropylacrylamide-co-butyl acrylate), poly(N-isopropylacrylamide-co-methacrylic acid), poly(N-isopropylacrylamide-co-methacrylic acid-co-octadecyl acrylate), and N-isopropylacrylamide.

The culture medium may be supplemented, for example, with a ROCK inhibitor so that the final concentration is from 1000 μmol/L to 0.1 μmol/L, from 100 μmol/L to 1 μmol/L, or from 5 μmol/L to 20 μmol/L. By adding the ROCK inhibitor to the culture medium, colony formation by stem cells is promoted.

The medium may, for example, be free of growth factors such as bFGF. Alternatively, the medium may contain growth factors such as bFGF at a low concentration of not more than 400 μg/L, not more than 100 μg/L, not more than 40 μg/L, or not more than 10 μg/L.

The medium may also be free of TGF-β, or may contain TGF-β at a low concentration of not more than 600 ng/L, not more than 300 ng/L, or not more than 100 ng/L.

The culture medium of stem cells may be cryopreserved. The supernatant of the stem cell culture medium contains lipids. Examples of lipids include simple lipids and complex lipids. Examples of simple lipids include neutral fats, cholesterol esters, and waxes. Neutral fats are esters of alcohols and fatty acids. Cholesterol esters are esters of cholesterol and fatty acids. Waxes are esters of higher alcohols and fatty acids. Examples of complex lipids include phospholipids, glycolipids, and derived lipids. Phospholipids are lipids containing a phosphate group. Examples of phospholipids include diglycerides and triglycerides. Glycolipids are lipids containing sugar moieties. The lipids may also be lipid-soluble hydrolysates of simple lipids or complex lipids. Examples of such lipids include fatty acids and cholesterol. When the composition according to the embodiment is applied to the skin, the phospholipids contained in the supernatant of the stem cell culture medium are incorporated into intercellular lipids and form a lamellar structure. The phospholipids contained in the supernatant of the stem cell culture medium may be incorporated into liposomes.

For example, prior to being cultured, stem cells may be dissociated into single cells, and the dissociated single cells may be introduced into a culture medium. The single cells proliferate while maintaining clonality and an undifferentiated state, and form colonies in the medium. Whether the stem cells have maintained an undifferentiated state can be confirmed by examining whether the cells express undifferentiated cell markers. For example, the stem cells exhibit a positive rate for NANOG, TRA-1-60, SSEA3, SSEA4, TRA-1-81, or OCT3/4 of 30% or more, 50% or more, and preferably 80% or more.

The temperature for maintaining and culturing stem cells is, for example, 37 °C. The concentration of carbon dioxide during maintenance culture of the stem cells is, for example, 5%. The duration of the maintenance culture of the stem cells is, for example, from 1 day to 90 days, from 2 days to 60 days, from 3 days to 40 days, from 5 days to 30 days, or from 7 days to 21 days. The supernatant of the culture medium in which stem cells have been maintained in an undifferentiated state is obtained by removing the stem cells by, for example, filtration and centrifugation. A filter may be used at the filtration. The pore size of the filter is, for example, from 0.1 μm to 0.5 μm. In the case of suspension culture of the stem cells, the medium may be stirred at a speed at which 10% or more, 20% or more and 60% or less, or 20% or more and 50% or less of the cell clumps settle. The density of the clumps in the medium is, for example, from 50 clumps/mL to 2500 clumps/mL, from 300 clumps/mL to 1200 clumps/mL, from 200 clumps/mL to 1100 clumps/mL, or from 100 clumps/mL to 1000 clumps/mL.

Induced pluripotent stem (iPS) cells are induced, for example, by introducing reprogramming factors such as OCT3/4, KLF4, c-MYC, and SOX2 into somatic cells, such as differentiated cells including blood cells, urine-derived cells, fibroblasts of skin cells, dental pulp stem cells, and hair follicle cells. The iPS cells may also be induced from CD3-positive T cells, CD16-positive monocytes, CD68-positive macrophages, or CD34-or CD43-positive hematopoietic stem cells. Examples of blood cells include umbilical cord blood-derived cells, peripheral blood-derived cells, and mononuclear cells. Preferably, the iPS cells are induced by methods that do not cause gene integration, such as a method using Sendai virus or an RNA lipofection method. The induction of iPS cells may be referred to as reprogramming, nuclear reprogramming, reversion, transdifferentiation or lineage reprogramming, or cell fate reprogramming. The pluripotent stem cells may be induced under three-dimensional culture conditions such as suspension culture. In the three-dimensional culture, either a gel medium or a liquid medium may be used. As the culture medium used for induction, a human ES/iPS medium such as TeSR2 (STEMCELL Technologies) may be used, for example.

An exosome is a particulate substance secreted from cells. The exosome contains, on its surface, lipids and proteins derived from the cell membrane. In addition, the exosome contains, in its interior, nucleic acids and proteins derived from the interior of the cell. Exosomes derived from stem cells may be contained in the supernatant of the stem cell culture medium. The exosomes derived from stem cells may also be isolated from the culture medium of the stem cells. Examples of methods for isolating exosomes include ultracentrifugation, size-exclusion chromatography, and affinity purification methods targeting surface molecules of the exosomes. For example, exosomes may be purified using the MagCapture™ Exosome Isolation Kit (FUJIFILM Wako Pure Chemical Corporation).

At least a part of the stem cell may be an entire stem cell or a portion of the stem cell.

An extract of the stem cell may be an extract derived from the entire stem cell or from a part of the stem cell. The extract of the stem cell may be in liquid form; that is, the extract may be an extract solution. The extract of the stem cell may also be in the form of a stem cell paste. The stem cell paste may be obtained by grinding the stem cell. The extract of the stem cell may be a freeze-dried product. The extract of the stem cell may also be in powder form. Alternatively, the extract of the stem cell may be a lysate of the stem cell.

Examples of the liposomal component include a lipid. Examples of the lipid include a phospholipid. Examples of the phospholipid include a glycerophospholipid and a sphingolipid. Examples of the glycerophospholipid include phosphatidylcholine (lecithin), phosphatidylinositol, plasmalogen, and ether-linked phospholipid. The lecithin may be hydrogenated lecithin. Examples of sphingolipid include sphingosine, ceramide, sphingophospholipid, and sphingoglycolipid. The lipid may be a hydrogenated lipid or a hydrogenated phospholipid. The hydrogenated phospholipid may be hydrogenated lecithin.

The liposomal component may include isopentyldiol, glycerin, and sterol. The sterol may be soybean sterol. The liposomal components may constitute a liposome.

For example, the liposomal components may be contained in a solution containing a supernatant of a stem cell culture medium, a supernatant of a culture medium used when reprogramming somatic cells, a solution containing an exosome derived from stem cells, a solution containing at least a portion of stem cells, or a solution containing an extract of stem cells. In the composition, the liposomal components; and the solution containing a supernatant of a stem cell culture medium, the solution containing a supernatant of a culture medium used when reprogramming somatic cells, the solution containing the exosome derived from stem cells, the solution containing at least a portion of stem cells, or the solution containing an extract of stem cells, may be separated from each other.

When dispersing phospholipids in an aqueous solution, highly water-soluble phospholipids, such as hydrogenated phospholipids, may be directly dispersed in hot water. Phospholipids having low water solubility may be dissolved in a polyhydric alcohol solvent and then dispersed in water or hot water. Alternatively, the phospholipids may be dissolved in an organic solvent in a container, the organic solvent may be evaporated to form a thin film on the bottom surface of the container, and the resulting thin film may then be dispersed in water or hot water. Furthermore, a portion of water may be added to the organic solvent before evaporation, and the above operation may be carried out thereafter.

A liposome is a closed vesicle formed by a lipid bilayer composed of lipids such as phospholipids. The liposome may have a particle diameter of, for example, from 20 nm to 400 nm, from 50 nm to 300 nm, or from 60 nm to 250 nm. The liposome may be formed, for example, by dispersing lipids such as phospholipids and/or sterols in a polyhydric alcohol and subjecting the resulting dispersion to high-pressure dispersion treatment using a high-pressure emulsifier or a homomixer. The liposome may include phytomoist lipids, which are nano-sized capsules in which cosmetic ingredients are encapsulated within a lipid membrane. The composition may also include phytomoist lipids that do not encapsulate any cosmetic ingredients, and the cosmetic ingredients separately.

For example, the liposome may be contained in the solution containing the supernatant of the stem cell culture medium, the supernatant of the culture medium used when reprogramming the somatic cells, the secretion derived from the stem cells or the somatic cells into which the reprogramming factors have been introduced, the solution containing the exosome derived from the stem cells, the solution containing at least a portion of the stem cells, or the solution containing the extract of stem cells. The interior of the liposome may contain, as a cosmetic ingredient, or may not contain, the solution containing the supernatant of a stem cell culture medium, the solution containing the supernatant of the culture medium used when reprogramming the somatic cells, the solution containing the exosome derived from the stem cells, the solution containing at least a portion of the stem cells, or the solution containing the extract of stem cells.

Even when the interior of the liposome does not contain the solution containing the supernatant of the stem cell culture medium, the solution containing the supernatant of the culture medium used when reprogramming the somatic cells, the solution containing the secretion derived from the stem cells or from the somatic cells into which the reprogramming factors have been introduced, the solution containing the exosome derived from the stem cells, the solution containing at least a portion of the stem cells, or the solution containing the extract of stem cells, the coexistence of such a solution with the liposome enhances the permeability of the solution into the skin.

While not wishing to be bound by theory, it is believed that, as the liposome penetrates the skin while improving the condition of the skin, the solution containing the supernatant of the stem cell culture medium, the solution containing the supernatant of the culture medium used when reprogramming the somatic cells, the solution containing the secretion derived from the stem cells or from the somatic cells into which the reprogramming factors have been introduced, the solution containing the exosome derived from the stem cells, the solution containing at least a portion of the stem cells, or the solution containing the extract of stem cells, which is present in the vicinity of the liposome, also becomes more likely to penetrate the skin simultaneously.

The content of phospholipids in the composition according to the embodiment is, for example, from 0.01% to 25%. By setting the phospholipid content to 0.01% or more, the composition is more likely to exhibit an enhanced skin permeability effect. Additionally, by setting the phospholipid content to 25% or less, gelation of the composition can be suppressed.

In the case where the liposome is composed of hydrogenated phospholipids and a liquid hydrocarbon, the mass ratio of the hydrogenated phospholipids, the liquid hydrocarbon, and the aqueous phase containing the culture supernatant is, for example, from 0.05 to 15:from 0.01 to 5:from 30.5 to 99.96.

The composition according to the embodiment may contain, for example: (a) from 0.1 mass % to 5 mass % of phospholipids; (b) from 0.1 mass % to 5 mass % of cholesterol and/or a derivative thereof; and (c) from 5 mass % to 40 mass % of glycerin and/or 1,3-butylene glycol, wherein the ratio (a):(b) may be from 1:0.1 to 1:1.

The liposome may contain a phospholipid containing iodine and phosphatidylcholine, and a poorly water-soluble coenzyme Q10.

The liposome may also be produced by uniformly dissolving carotenoids, sterols, and phospholipids in an organic solvent and removing the solvent. By heating and dispersing a complex of carotenoids, sterols, and phospholipids in a polyhydric alcohol and stirring with a homomixer or the like, poorly water-soluble carotenoids can be stably incorporated into the liposome.

In the case where the solution containing the supernatant of the stem cell culture medium, the solution containing the supernatant of the culture medium used when reprogramming the somatic cells, the solution containing the secretion derived from the stem cells or from the somatic cells into which the reprogramming factors have been introduced, the solution containing the exosome derived from the stem cells, the solution containing at least a portion of the stem cells, or the a solution containing the extract of stem cells is encapsulated in a liposome, lipids may be dispersed in such a solution. By subjecting the resulting dispersion to high-pressure dispersion treatment using a high-pressure emulsifier or homomixer, the liposome encapsulating the solution containing the supernatant of the stem cell culture medium, the solution containing the supernatant of the culture medium used when reprogramming the somatic cells, the solution containing the secretion derived from the stem cells or from the somatic cells into which the reprogramming factors have been introduced, the solution containing the exosome derived from stem cells, the solution containing at least a portion of the stem cells, or the solution containing the extract of stem cells can be formed.

The concentration of liposomes in the composition is, for example, from 0.05 mass % to 80 mass %, from 0.1 mass % to 60 mass %, from 0.2 mass % to 50 mass %, or from 0.5 mass % to 25 mass %. The concentration of liposomal components in the composition is, for example, from 0.5 mass % to 15 mass %, from 1 mass % to 10 mass %, or from 1 mass % to 8 mass %. The concentration of the supernatant of the stem cell culture medium in the composition is, for example, 0.1 mass % or more, 1.0 mass % or more, 3.0 mass % or more, 5.0 mass % or more, or 25 mass % or more, and 99.0 mass % or less, 80.0 mass % or less, 50.0 mass % or less, 20.0 mass % or less, or less than these values. The concentration of the secretion derived from stem cells or from somatic cells into which reprogramming factors have been introduced is, for example, 0.1 mass % or more, 1.0 mass % or more, 3.0 mass % or more, 5.0 mass % or more, or 25 mass % or more, and 99.0 mass % or less, 80.0 mass % or less, 50.0 mass % or less, 20.0 mass % or less, or less than these values. The concentration of exosomes derived from stem cells in the composition is, for example, 0.1 mass % or more, 1.0 mass % or more, 3.0 mass % or more, 5.0 mass % or more, or 25 mass % or more, and 99.0 mass % or less, 80.0 mass % or less, 50.0 mass % or less, 20.0 mass % or less, or less than these values. The concentration of at least a portion of stem cells in the composition is, for example, 0.1 mass % or more, 1.0 mass % or more, 3.0 mass % or more, 5.0 mass % or more, or 25 mass % or more, and 99.0 mass % or less, 80.0 mass % or less, 50.0 mass % or less, 20.0 mass % or less, or less than these values. The concentration of stem cell extract in the composition is, for example, 0.1 mass % or more, 1.0 mass % or more, 3.0 mass % or more, 5.0 mass % or more, or 25 mass % or more, and 99.0 mass % or less, 80.0 mass % or less, 50.0 mass % or less, 20.0 mass % or less, or less than these values.

The composition according to the embodiment may be a topical skin composition. The composition according to the embodiment may also be a therapeutic agent for skin diseases. Examples of diseases that can be treated with the skin disease therapeutic agent according to the embodiment include acne vulgaris, psoriasis vulgaris, keloid, seborrheic dermatitis, contact dermatitis, atopic dermatitis, atopic dry dermatitis, dermatoporosis, actinic elastosis, actinic keratosis, blepharoptosis, alopecia areata, hair loss, eyelash hypotrichosis, melasma, senile lentigines, heat rash, freckles, acquired bilateral nevus of Ota-like macules, seborrheic keratosis, skin diseases caused by progeria, and herpes simplex.

Examples of conditions that may be ameliorated or alleviated by the composition according to the embodiment include spots, freckles, wrinkles, sagging, roughness, loss of skin firmness, dullness, sensitive skin, dry skin, and thinning hair. Effects of the composition according to the embodiment include conditioning the skin, improving skin texture, maintaining healthy skin, preventing skin roughness, tightening the skin, moisturizing the skin, supplementing and retaining moisture and oil in the skin, maintaining skin elasticity, protecting the skin, preventing dryness, softening the skin, improving skin firmness, imparting luster to the skin, smoothing the skin, reducing the appearance of spots, suppressing the formation of wrinkles, and brightening the skin. Furthermore, effects of the composition on the scalp or hair may include maintaining a healthy scalp, promoting hair growth, preventing hair thinning, preventing itching, preventing hair loss, stimulating hair production, promoting hair regeneration, preventing hair loss after illness or childbirth, and nourishing the hair.

The composition according to the embodiment may be a wound healing agent, an epidermal cell proliferation promoter, an epidermal turnover accelerator, a hair growth agent, a hair growth promoter, a hair nourishing agent, or a therapeutic agent for eyelash hypotrichosis. The composition according to the embodiment may also be a collagen production enhancer, a hyaluronic acid production enhancer, a hair growth agent, a fibroblast growth factor (FGF) family production enhancer, or a vascular endothelial growth factor (VEGF) production enhancer.

In hair growth, matrix cells located in the hair root undergo cell division, and the resulting cells constitute the hair shaft. Hair growth follows a cycle known as the hair cycle, which consists of the anagen (growth phase), catagen (regression phase), and telogen (resting phase) phases in a repetitive sequence. Dermal papilla cells influence the proliferation and differentiation of hair follicle epithelial stem cells through the production and release of growth factors, thereby regulating the hair cycle. It is believed that activation of dermal papilla cells and matrix cells contributes to the mechanism of hair growth. In addition, active vascular remodeling occurs in the hair follicle in accordance with the hair cycle; however, impaired angiogenesis during this process may lead to insufficient supply of nutrients and oxygen required for hair formation. A deficiency in blood flow from the capillary network of the hair follicle is believed to be associated with the pathology of androgenetic alopecia (AGA).

With respect to genes expressed in dermal papilla cells and their relation to hair growth and hair regeneration, the following has been reported. Specifically, growth factors secreted by dermal papilla cells toward matrix cells include, for example, fibroblast growth factor-7 (FGF-7) and insulin-like growth factor-1 (IGF-1). These genes are known to play a role in maintaining hair follicle growth. Vascular endothelial growth factor (VEGF) is secreted by dermal papilla cells and contributes to the angiogenesis of follicular blood vessels. In addition, VEGF exhibits an autocrine effect to promote the proliferation of dermal papilla cells. However, the expression level of VEGF decreases as the hair cycle transitions from the anagen phase to the catagen phase. The expression of the VEGF gene is known to be reduced in hair tissues affected by androgenetic alopecia (AGA). VEGF-B binds competitively to VEGF receptor-1 (VEGFR-1), which is a receptor for VEGF. Although VEGF-B exhibits endothelial cell proliferative and permeability-enhancing activities, its effects in hair follicles remain unclear.

The composition according to the embodiment may act directly on the dermal papilla, increase the production of fibroblast growth factor-7 (FGF-7), which is a hair growth-promoting factor, thereby promoting hair growth and prolonging the anagen phase of the hair cycle, resulting in the development of thicker and stronger hair from fine and weak hair. The composition may also enhance the expression of vascular endothelial growth factor (VEGF), which is secreted from dermal papilla cells and contributes to the angiogenesis of follicular blood vessels, and further exhibits an autocrine effect that promotes proliferation of dermal papilla cells.

When the composition according to the embodiment is used as a hair growth promoter or hair regrowth agent, it may further contain other active ingredients such as minoxidil, swertia extract, pantothenyl ethyl ether, tocopheryl acetate, dipotassium glycyrrhizinate, and adenosine.

Examples of wounds that can be treated with the wound healing agent according to the embodiment include burns, abrasions, lacerations, contusions, surgical sutured wounds, pressure ulcers, and skin defects.

The composition according to the embodiment may be a cytoprotective agent that protects cells from stress. In addition, the composition according to the embodiment may be a cell survival-enhancing agent that stabilizes cells under stress and, for example, improves cell viability. The stressed cells may include, for example, fibroblasts, epidermal cells, and dermal papilla cells, but are not limited thereto and may be any type of cell. Alternatively, the composition according to the embodiment may be a biopolymer-protecting agent that protects at least one biological substance selected from the group consisting of nucleic acids, proteins, protein complexes, lipoproteins, ribosomes, and biological membranes from stress. Here, the biological membranes include cell membranes.

The composition according to the embodiment contains an effective amount of at least one selected from the group consisting of the supernatant of the stem cell culture medium, the supernatant of the culture medium used when reprogramming the somatic cells, the exosome derived from the stem cells, at least a portion of the stem cells, and the extract of stem cells. Here, the term “effective amount” refers to an amount sufficient to exhibit a desired effect as a pharmaceutical or cosmetic composition. The effective amount may be appropriately determined depending on factors such as the age of the subject, the target condition, the presence or absence of other active ingredients, and the amount of other components included in the composition.

The composition according to the embodiment may contain pharmaceutically acceptable carriers, excipients, disintegrants, buffering agents, emulsifiers, suspending agents, analgesics, stabilizers, preservatives, antimicrobial agents, and physiological saline.

Examples of excipients include lactose, starch, sorbitol, D-mannitol, and sucrose. Examples of disintegrants include carboxymethylcellulose and calcium carbonate. Examples of buffering agents include phosphates, citrates, and acetates. Examples of emulsifiers include gum arabic, sodium alginate, and tragacanth.

Examples of suspending agents include glyceryl monostearate, aluminum monostearate, methylcellulose, carboxymethylcellulose, hydroxypropylcellulose, and sodium lauryl sulfate. Examples of analgesics include benzyl alcohol, chlorobutanol, and sorbitol. Examples of stabilizers include propylene glycol and ascorbic acid. Examples of preservatives include phenol, benzalkonium chloride, benzyl alcohol, chlorobutanol, and methylparaben. Examples of antimicrobial agents include benzalkonium chloride, paraoxybenzoic acid, and chlorobutanol.

The composition according to the embodiment may further contain, to the extent that the purpose of the composition is achieved, one or more of the following: water; alcohols; surfactants (including cationic, anionic, nonionic, and amphoteric surfactants); humectants (such as glycerin, 1,3-butylene glycol, propylene glycol, propanediol, pentanediol, polyquaterniums, amino acids, urea, pyrrolidone carboxylates, nucleic acids, monosaccharides, oligosaccharides, and derivatives thereof); thickeners (such as polysaccharides, polyacrylates, carboxyvinyl polymers, polyvinylpyrrolidone, polyvinyl alcohol, chitin, chitosan, alginic acid, carrageenan, xanthan gum, methylcellulose, and derivatives thereof); waxes; petrolatum; saturated and unsaturated hydrocarbon fatty acids; silicone oils and derivatives thereof; triglycerides such as glyceryl tri(caprylate/caprate) and glyceryl trioctanoate; ester oils such as isopropyl stearate; natural oils and fats such as olive oil, camellia oil, avocado oil, almond oil, cacao butter, evening primrose oil, grape seed oil, macadamia nut oil, eucalyptus oil, rosehip oil, squalane, orange roughy oil, lanolin, and ceramides; preservatives (such as oxybenzoate derivatives, dehydroacetic acid salts, photosensitizers, sorbic acid, and phenoxyethanol, including derivatives thereof); bactericides (such as sulfur, triclocarbanilide, salicylic acid, zinc pyrithione, hinokitiol, and derivatives thereof); ultraviolet absorbers (such as para-aminobenzoic acid, methoxycinnamic acid, and derivatives thereof); anti-inflammatory agents (such as allantoin, bisabolol, ε-aminocaproic acid, acetylphenethylcysteine, glycyrrhizic acid, and derivatives thereof); antioxidants (such as tocopherol, BHA, BHT, astaxanthin, and derivatives thereof); chelating agents (such as edetate and hydroxyethanediphosphonic acid, and derivatives thereof); animal and plant extracts (such as extracts or fermented products of Angelica keiskei, aloe, Fructus Rosae Multiflorae, Scutellaria, Phellodendron bark, seaweed, Chinese quince, chamomile, licorice, kiwi, cucumber, mulberry, birch, Japanese angelica, garlic, peony, hop, horse chestnut, lavender, rosemary, eucalyptus, milk, various peptides, placenta, royal jelly, Euglena extract, hydrolyzed Euglena extract, and Euglena oil); pH adjusters (such as inorganic acids, inorganic acid salts, organic acids, organic acid salts, and derivatives thereof); vitamins (such as vitamin A group, vitamin B group, vitamin C, vitamin D group, ubiquinone, nicotinamide, and derivatives thereof); fermentation products of yeast, koji mold, and lactic acid bacteria; Galactomyces culture filtrate; whitening agents (such as tranexamic acid, cetyl tranexamic acid hydrochloride, 4-n-butylresorcinol, arbutin, kojic acid, ellagic acid, licorice flavonoids, niacinamide, and vitamin C derivatives); ceramides and ceramide derivatives; anti-wrinkle agents (such as retinol, retinal, and their derivatives, nicotinamide, oligopeptides and derivatives thereof, and natural or synthetic ingredients having neutrophil elastase inhibitory activity and MMP-1 or MMP-2 inhibitory activity); and powders such as titanium oxide, talc, mica, silica, zinc oxide, iron oxide, silicone, and processed forms thereof.

The composition according to the embodiment can be produced by methods that are well known and routinely employed in the relevant technical field.

In the supernatant of a culture medium of stem cells included in the composition according to the embodiment, the stem cells may be derived from the human subject to whom the composition is to be administered. For example, cells may be collected from the subject, the collected cells may be reprogrammed to induce pluripotent stem cells, and the supernatant of the culture medium of the induced pluripotent stem cells may be included in the composition according to the embodiment and provided to the subject.

In the supernatant of the medium used when reprogramming somatic cells included in the composition according to the embodiment, the somatic cells may be derived from the human subject to whom the composition is to be administered. For example, somatic cells may be collected from the subject, and the supernatant of the culture medium used when reprogramming the collected cells to induce pluripotent stem cells may be included in the composition according to the embodiment and provided to the subject.

In the exosome derived from stem cells included in the composition according to the embodiment, the stem cells may be derived from the human subject to whom the composition is to be administered. For example, cells may be collected from the subject, the collected cells may be reprogrammed to induce pluripotent stem cells, and exosomes recovered from the culture medium of the induced pluripotent stem cells may be included in the composition according to the embodiment and provided to the subject. The method for recovering exosomes from the culture medium is not particularly limited. For example, exosomes may be recovered by centrifuging the culture medium using ultracentrifugation or density gradient centrifugation. Alternatively, exosomes may be recovered using proteins that are specifically expressed on exosomes. For example, phosphatidylserine is expressed on the membrane surface of exosomes. The exosome receptor Tim4, which is expressed on macrophages, binds phosphatidylserine in a calcium-dependent manner. Therefore, magnetic beads having the extracellular domain of Tim4 bound thereto may be prepared, and exosomes may be immobilized on the surface of the magnetic beads via the extracellular domain of Tim4.

Subsequently, the exosomes can be eluted from the magnetic beads using a chelating agent, thereby enabling recovery and isolation of the exosomes from the culture medium. Alternatively, exosomes may be recovered by coprecipitation with polyethylene glycol (PEG). Furthermore, exosomes may be isolated from serum or culture supernatant by immunoprecipitation using antibodies that specifically recognize CD9, CD63, or CD81.

In at least a portion of the stem cells included in the composition according to the embodiment, the stem cells may be derived from the human subject to whom the composition is to be administered. For example, cells may be collected from the subject, the collected cells may be reprogrammed to induce pluripotent stem cells, and at least a portion of the induced pluripotent stem cells may be included in the composition according to the embodiment and provided to the subject.

In the extract of stem cells included in the composition according to the embodiment, the stem cells may be derived from the human subject to whom the composition is to be administered. For example, cells may be collected from the subject, the collected cells may be reprogrammed to induce pluripotent stem cells, and the extract of the induced pluripotent stem cells may be included in the composition according to the embodiment and provided to the subject.

The composition according to the embodiment may be in the form of an injectable formulation or an infusion formulation.

The pharmaceutical and/or cosmetic composition according to the embodiment contains the secretion from the stem cells or from the somatic cells into which the reprogramming factors have been introduced. The pharmaceutical composition may be an over-the-counter drug or a quasi-drug.

The pharmaceutical and/or cosmetic composition containing the secretion from the stem cells or from the somatic cells into which the reprogramming factors have been introduced may be, for example, a lipolytic agent, an anti-obesity agent, an agent for ameliorating or preventing obesity, a lipolysis-promoting agent, a dietary agent, a therapeutic or prophylactic agent for diabetes, a prostacyclin production enhancer, a cyclic adenosine monophosphate (cAMP) production enhancer, a platelet aggregation inhibitor, a vasodilator, a therapeutic or prophylactic agent for hypertension, a therapeutic or prophylactic agent for arteriosclerosis, a vascular endothelial cell proliferation inhibitor, a hyaluronic acid production enhancer, a cartilage cell hyaluronic acid production enhancer, a cartilage cell proliferation promoter, a therapeutic or prophylactic agent for cartilage tissue injury, a therapeutic or prophylactic agent for joint disorders, a therapeutic or prophylactic agent for joint pain, a cartilage cell viability enhancer, a therapeutic or prophylactic agent for lifestyle-related diseases, a therapeutic or prophylactic agent for adult diseases, an amyloid β inhibitor, a therapeutic or prophylactic agent for neurological disorders, a therapeutic or prophylactic agent for dementia, a neuronal cell death inhibitor, a therapeutic or prophylactic agent for reduction in neuronal cell number, a collagen production enhancer, an agent for preventing or ameliorating formation of skin spots, wrinkles, or sagging, a fibroblast growth factor family production enhancer, a vascular endothelial growth factor production enhancer, a hair growth agent, a hair restoration agent, a fibroblast proliferation promoter, or a wound healing agent.

The stem cells may be, for example, pluripotent stem cells such as induced pluripotent stem (iPS) cells or embryonic stem(ES) cells. The stem cells may also be totipotent stem cells or multipotent stem cells. The stem cells may be, for example, reprogrammed cells. The multipotent stem cells may be positive for at least one of CD73, CD90, CD105, CD34, CD45, EpCAM, EGFR, cadherin, CD133/PROM1, or nestin. The stem cells may also include somatic stem cells or progenitor cells induced from pluripotent stem cells. Examples of somatic stem cells include mesenchymal stem cells, hematopoietic stem cells, and neural stem cells. The stem cells may be cultured in two-dimensional or three-dimensional systems. The stem cells may consist solely of iPS cells and may exclude other types of stem cells. The stem cells may also be derived from the subject to whom the pharmaceutical or cosmetic composition is to be administered.

The secretion from the stem cells or from the somatic cells into which the reprogramming factors have been introduced may be, for example, extracellular vesicles. The extracellular vesicles are vesicles having a lipid bilayer membrane structure, and are also referred to as membrane vesicles. Examples of extracellular vesicles include exosomes and microvesicles. Exosomes have a diameter of about 30 nm to 150 nm. Exosomes are formed by inward budding of the membrane of late endosomes, which then fuse with the plasma membrane to form complete particles and are secreted extracellularly by exocytosis. Microvesicles have a diameter of about 100 nm to 1000 nm and are generated by outward budding and separation of the plasma membrane.

The exosome secretion process will be further described. When endocytosis occurs in a cell, early endosomes are first formed, followed by late endosomes. During this process, vesicles that are formed by inward budding of the endosomal membrane are referred to as intraluminal vesicles (ILVs). An endosome containing a plurality of intraluminal vesicles is referred to as a multivesicular body (MVB). When the multivesicular body fuses with the plasma membrane, the intraluminal vesicles within the multivesicular body are secreted extracellularly through exocytosis. The intraluminal vesicles released into the extracellular space are referred to as exosomes. It has been reported that the secretion process involves ceramides and components of the endosomal sorting complex required for transport (ESCRT), such as Hrs, which play important roles in vesicle budding into the endosomal membrane, and members of the RAB family, such as RAB11 and RAB35, which are small GTP-binding proteins involved in intracellular transport of vesicles. In addition, it has been reported that SNARE, a membrane-associated protein, is involved in the fusion of multivesicular bodies with the plasma membrane and the exocytic release of exosomes.

The secretion from the stem cells or from the somatic cells into which the reprogramming factors have been introduced may be, for example, ectosomes, secretory microvesicles, oncosomes, or prostasomes.

The secretion from the stem cells or from the somatic cells into which the reprogramming factors have been introduced may be, for example, peptides, proteins, growth factors, proliferation factors, cytokines, or hormones. The proteins may have, for example, a molecular weight of 3 kDa or more, 10 kDa or more, or 20 kDa or more.

The pharmaceutical and/or cosmetic composition according to the embodiment may contain isolated secretions from the stem cells or from the somatic cells into which the reprogramming factors have been introduced. Alternatively, the pharmaceutical and/or cosmetic composition according to the embodiment may contain concentrated secretions from such cells. Examples of methods for isolating or concentrating the secretions include lyophilization (freeze-drying), ultrafiltration, precipitation and resuspension, and gel filtration. Examples of protein precipitation methods include ammonium sulfate precipitation, trichloroacetic acid precipitation, acetone precipitation, and methanol precipitation in the presence of ammonium acetate.

Examples of marker proteins for extracellular vesicles include tetraspanin family proteins (CD9, CD63, CD81), integrin family proteins (ITGA, ITGB), ESCRT complex-associated proteins (TSG101, ALIX), lipid raft markers (Flotillin-1/2), and heat shock proteins (HSP70). Therefore, extracellular vesicles may be isolated using antibodies that specifically recognize these marker proteins.

The secretion may be recovered from the supernatant of the culture medium of the stem cells or the somatic cells into which the reprogramming factors have been introduced. The secretion may be recovered from the supernatant of a culture medium during maintenance culture of the stem cells. The secretion may also be recovered from the supernatant of a culture medium during the reprogramming process of somatic cells into which reprogramming factors have been introduced. The secretion may be recovered from at least a portion of the stem cells or somatic cells into which the reprogramming factors have been introduced. The secretion may also be recovered from an extract of such stem cells or reprogrammed somatic cells.

The inventors of the present invention have found, for the first time, that the secretion from the stem cells or from the somatic cells into which the reprogramming factors have been introduced is capable of degrading fat. Accordingly, the secretion from the stem cells or the reprogrammed somatic cells may be used as a lipolytic agent, an anti-obesity agent, an agent for ameliorating obesity, a lipolysis-promoting agent, or a dietary agent. It is also known that fat levels in the blood are elevated in diabetes. Therefore, the secretion from the stem cells or from the somatic cells into which the reprogramming factors have been introduced may be used as a therapeutic or prophylactic agent for diabetes. The fat may be, for example, neutral fat (triglycerides). The diabetes may be type 1 diabetes, type 2 diabetes, other specific types due to identified mechanisms or diseases, or gestational diabetes.

The inventors of the present invention have discovered, for the first time, that the secretion from the stem cells or from the somatic cells into which the reprogramming factors have been introduced promotes the production of prostacyclin by endothelial cells. Accordingly, the secretion from the stem cells or from the somatic cells into which the reprogramming factors have been introduced can be used as a prostacyclin production enhancer. Prostacyclin is also referred to as prostaglandin I₂ (PGI₂). Prostacyclin activates adenylate cyclase and promotes the production of cyclic adenosine monophosphate (cAMP). Therefore, the secretion from the stem cells or from the somatic cells into which the reprogramming factors have been introduced can also be used as an adenylate cyclase activator or a cyclic adenosine monophosphate (cAMP) production enhancer.

Prostacyclin also exhibits platelet aggregation inhibitory activity and vasodilatory activity on vascular smooth muscle. Accordingly, the secretion from the stem cells or from the somatic cells into which the reprogramming factors have been introduced can be used as a platelet aggregation inhibitor, a vasodilator, a therapeutic or prophylactic agent for hypertension, and a therapeutic or prophylactic agent for arteriosclerosis. Furthermore, prostacyclin inhibits the proliferation of vascular endothelial cells. Therefore, the secretion from the stem cells or from the somatic cells into which the reprogramming factors have been introduced can also be used as a vascular endothelial cell proliferation inhibitor.

The inventors of the present invention have discovered, for the first time, that the secretion from the stem cells or from the somatic cells into which the reprogramming factors have been introduced promotes the production of hyaluronic acid by chondrocytes and fibroblasts. Accordingly, the secretion from the stem cells or from the somatic cells into which the reprogramming factors have been introduced can be used as a hyaluronic acid production enhancer for chondrocytes and fibroblasts. In addition, hyaluronic acid protects cartilage and exerts lubricating, cushioning, and anti-inflammatory effects on joints. Therefore, the secretion from the stem cells or from the somatic cells into which the reprogramming factors have been introduced can also be used as a chondrocyte proliferation promoter, a therapeutic or prophylactic agent for cartilage tissue injury, and a therapeutic or prophylactic agent for joint pain.

The inventors of the present invention have discovered, for the first time, that the secretion from the stem cells or from the somatic cells into which the reprogramming factors have been introduced activates chondrocytes, enhances their viability, promotes their proliferation, and rejuvenates the chondrocytes. Accordingly, the secretion from the stem cells or from the somatic cells into which the reprogramming factors have been introduced can be used as a chondrocyte activator, a chondrocyte viability enhancer, a chondrocyte proliferation promoter, and a chondrocyte rejuvenation agent.

The inventors of the present invention have discovered, for the first time, that the secretion from the stem cells or from the somatic cells into which the reprogramming factors have been introduced promotes the production of collagen by skin cells such as fibroblasts, epidermal cells, and dermal cells. Accordingly, the secretion from the stem cells or from the somatic cells into which the reprogramming factors have been introduced can be used as a collagen production enhancer. Furthermore, collagen has the effect of preventing and ameliorating the formation of skin spots, wrinkles, and sagging. Therefore, the secretion from the stem cells or from the somatic cells into which the reprogramming factors have been introduced can also be used as an agent for preventing or ameliorating at least one of skin spots, wrinkles, or sagging.

The inventors of the present invention have discovered, for the first time, that the secretion from the stem cells or from the somatic cells into which the reprogramming factors have been introduced promotes the production of FGF7 and VEGF by hair papilla cells. Accordingly, the secretion from the stem cells or from the somatic cells into which the reprogramming factors have been introduced can be used as a fibroblast growth factor (FGF) family production enhancer and a vascular endothelial growth factor (VEGF) production enhancer. Furthermore, FGF7 and VEGF exhibit hair growth-promoting and hair regeneration effects. Therefore, the secretion from the stem cells or from the somatic cells into which the reprogramming factors have been introduced can be used as a hair growth agent and a hair restoration agent.

The inventors of the present invention have discovered, for the first time, that the secretion from the stem cells or from the somatic cells into which the reprogramming factors have been introduced promotes the proliferation of fibroblasts. Accordingly, the secretion from the stem cells or from the somatic cells into which the reprogramming factors have been introduced can be used as a fibroblast proliferation promoter. In addition, it is known that fibroblasts proliferate at wound sites to promote wound healing. Therefore, the secretion from the stem cells or from the somatic cells into which the reprogramming factors have been introduced can also be used as a wound healing agent.

Furthermore, anti-obesity effects, muscle mass-increasing effects, vasodilatory effects, and cartilage-protective effects are associated with the suppression of aging and contribute to the treatment and prevention of lifestyle-related diseases and adult-onset diseases. Accordingly, the secretion from the stem cells or from the somatic cells into which the reprogramming factors have been introduced can be used as an anti-aging agent, a therapeutic or prophylactic agent for lifestyle-related diseases, and/or a therapeutic or prophylactic agent for adult-onset diseases.

Amyloid beta (AB) is a relatively small protein (peptide) composed of approximately 40 amino acid residues and is known to accumulate in the brains of patients with Alzheimer's disease to form amyloid plaques. When amyloid beta accumulates in the extracellular space of neurons in the brain, the accumulation of amyloid beta induces the intracellular accumulation of tau protein. When the tau protein that has accumulated inside neurons aggregates, neurofibrillary tangles (NFTs) are formed. It is believed that the toxicity of accumulated amyloid beta and the accumulation of NFTs cause neuronal cell death, which leads to the onset of Alzheimer-type dementia.

In addition to Alzheimer's disease, dementia with Lewy bodies, vascular dementia, and frontotemporal dementia collectively account for approximately 90% of all dementia cases and are therefore referred to as the four major types of dementia. Although dementia with Lewy bodies, vascular dementia, and frontotemporal dementia are believed to develop through mechanisms different from those of Alzheimer's disease, it has also been reported that amyloid beta accumulation is associated with brain atrophy in dementia with Lewy bodies.

It is known that diabetes increases the risk of developing Alzheimer's disease. In diabetic patients with increased insulin resistance, insulin secretion is enhanced in order to lower blood glucose levels. The secreted insulin is degraded by insulin-degrading enzyme (IDE), which is also responsible for degrading amyloid beta. When insulin is present in excess, IDE is consumed for insulin degradation, thereby reducing its capacity to degrade amyloid beta. As a result, amyloid beta that is not degraded is thought to accumulate.

It is also known that individuals with Down syndrome have a relatively high risk of developing early-onset Alzheimer's disease. Down syndrome is caused by trisomy of chromosome 21, on which the gene encoding amyloid precursor protein (APP), the precursor of amyloid beta, is located. Therefore, it is believed that increased production of amyloid beta in individuals with Down syndrome, compared to healthy individuals, contributes to the elevated risk of developing early-onset Alzheimer's disease in such individuals.

Diseases caused by the accumulation of amyloid proteins are referred to as amyloidoses. Amyloidosis can be classified into systemic amyloidosis, in which amyloid accumulates in multiple organs and causes dysfunction, and localized amyloidosis, in which amyloid accumulates in a single organ and causes dysfunction. Alzheimer's disease, which is believed to result from the accumulation of amyloid beta, is a type of localized amyloidosis.

The inventors of the present invention have confirmed that the addition of amyloid beta to neuronal cells induces neuronal cell death, thereby verifying the neurotoxic effect of amyloid beta. On the other hand, the inventors have newly discovered that the neuronal cell death is suppressed when the secretion from the stem cells or from the somatic cells into which the reprogramming factors have been introduced is added to neuronal cells together with amyloid beta. Accordingly, the secretion from the stem cells or from the somatic cells into which the reprogramming factors have been introduced can be used as an inhibitor of protein accumulation, aggregation, or mislocalization, preferably as an amyloid beta inhibitor. As used herein, inhibition of amyloid beta includes inhibition of its accumulation, aggregation, or mislocalization, and also includes inhibition of the effects of amyloid beta, particularly its effects on neuronal cells.

As described above, Alzheimer's-type dementia is believed to be caused by the accumulation and aggregation of amyloid beta. Accordingly, the secretion from the stem cells or from the somatic cells into which the reprogramming factors have been introduced can be used as a therapeutic or prophylactic agent for dementia. The dementia may include Alzheimer's-type dementia and/or dementia with Lewy bodies. The dementia may also be dementia associated with diabetes or dementia occurring in individuals with Down syndrome.

Furthermore, since neuronal cell death is suppressed in the presence of the secretion from the stem cells or from the somatic cells into which the reprogramming factors have been introduced, such secretion can be used as a neuronal cell death inhibitor or as a therapeutic or prophylactic agent for neurological disorders. The neurological disorder may be one accompanied by the accumulation, aggregation, or mislocalization of proteins such as amyloid beta, and is preferably a neurological disorder caused by the accumulation or aggregation of amyloid beta. For example, it may be neurofibrillary tangle (NFT) formation.

Since the secretion from the stem cells or from the somatic cells into which the reprogramming factors have been introduced inhibits amyloid beta, it can be used as a therapeutic or prophylactic agent for amyloidosis. The amyloidosis may be systemic amyloidosis or localized amyloidosis. The amyloidosis may include, for example, Alzheimer's-type dementia.

The inventors of the present invention have discovered, for the first time, that when the secretion from the stem cells or from the somatic cells into which the reprogramming factors have been introduced is added to neuronal cells together with amyloid beta, the number of surviving neuronal cells increases compared to the case in which neither amyloid beta nor the secretion is added. Accordingly, the secretion from the stem cells or from the somatic cells into which the reprogramming factors have been introduced can be used as a neuronal cell survival enhancer or as a neuronal cell protector.

Since the secretion from the stem cells or from the somatic cells into which the reprogramming factors have been introduced suppresses neuronal cell death and/or enhances neuronal cell survival, such secretion can be used as a therapeutic or prophylactic agent for diseases caused by neuronal cell death. The neuronal cell death includes apoptosis. The diseases caused by neuronal cell death may include, for example, Alzheimer's disease, Huntington's disease, Parkinson's disease, and/or amyotrophic lateral sclerosis (ALS). In addition, the secretion from stem cells or from somatic cells into which reprogramming factors have been introduced can be used as a therapeutic or prophylactic agent for neuronal cell loss or neuronal cell death.

Furthermore, the suppression of neuronal cell death, enhancement of neuronal cell survival, or protection of neuronal cells may contribute to neuronal rejuvenation or prevention of neuronal aging. Accordingly, the secretion from the stem cells or from the somatic cells into which the reprogramming factors have been introduced can be used as a neuronal rejuvenation agent or an anti-neuronal aging agent.

The neuronal cells are not particularly limited and may include, for example, cerebral neurons, excitatory neurons, inhibitory neurons, motor neurons, peripheral neurons, and interneurons. The neuronal cells may be neurons that have been collected or may be neurons induced through differentiation from stem cells. The stem cells may include, for example, induced pluripotent stem (iPS) cells. The neuronal cells obtained by collection or differentiation may be positive for proteins such as vGlu (vesicular glutamate transporter), Satb2 (Special AT-rich sequence-binding protein 2), CTIP (C-terminal binding protein interacting protein), and/or Reelin (REELIN).

The pharmaceutical and cosmetic compositions according to the embodiment contain an effective amount of the secretion from the stem cells or from the somatic cells into which the reprogramming factors have been introduced. As used herein, the term “effective amount” refers to an amount sufficient to exert a beneficial effect as a pharmaceutical or cosmetic. The effective amount may be appropriately determined depending on the age of the patient, the target disease or condition, the presence or absence of other active ingredients, and the quantity of other components contained in the formulation.

The pharmaceutical and/or cosmetic compositions containing the secretion from the stem cells or from the somatic cells into which the reprogramming factors have been introduced, according to the embodiment, may further contain pharmaceutically or cosmetically acceptable carriers, excipients, disintegrants, buffering agents, emulsifiers, suspending agents, analgesics, stabilizers, preservatives, antimicrobial agents, and physiological saline. Examples of excipients include lactose, starch, sorbitol, D-mannitol, and sucrose. Examples of disintegrants include carboxymethylcellulose and calcium carbonate. Examples of buffering agents include phosphates, citrates, and acetates. Examples of emulsifiers include gum arabic, sodium alginate, and tragacanth.

The pharmaceutical and/or cosmetic compositions containing the secretion from the stem cells or from the somatic cells into which the reprogramming factors have been introduced, according to the embodiment, may further contain, to the extent necessary to achieve the purpose of the pharmaceutical or cosmetic composition, one or more of the following: water; alcohols; surfactants (including cationic, anionic, nonionic, and amphoteric surfactants); humectants (such as glycerin, 1,3-butylene glycol, propylene glycol, propanediol, pentanediol, polyquaternium, amino acids, urea, pyrrolidone carboxylates, nucleic acids, monosaccharides, oligosaccharides, and derivatives thereof); thickeners (such as polysaccharides, polyacrylic acid salts, carboxyvinyl polymers, polyvinylpyrrolidone, polyvinyl alcohol, chitin, chitosan, alginic acid, carrageenan, xanthan gum, methylcellulose, and derivatives thereof); waxes; petrolatum; saturated and unsaturated hydrocarbon fatty acids; silicone oils and their derivatives; triglycerides such as glyceryl tri(caprylate/caprate) and glyceryl trioctanoate; ester oils such as isopropyl stearate; and natural oils and fats including olive oil, camellia oil, avocado oil, almond oil, cacao butter, evening primrose oil, grape seed oil, macadamia nut oil, eucalyptus oil, rosehip oil, squalane, orange roughy oil, lanolin, and ceramides. The composition may also include preservatives (such as oxybenzoate derivatives, dehydroacetic acid salts, photosensitizers, sorbic acid, phenoxyethanol, and their derivatives); bactericides (such as sulfur, triclocarbanilide, salicylic acid, zinc pyrithione, hinokitiol, and their derivatives); ultraviolet absorbers (such as para-aminobenzoic acid, methoxycinnamic acid, and their derivatives); anti-inflammatory agents (such as allantoin, bisabolol, β-aminocaproic acid, acetylphenethylcysteine, glycyrrhizic acid, and their derivatives); antioxidants (such as tocopherol, BHA, BHT, astaxanthin, and their derivatives); chelating agents (such as edetates, hydroxyethanediphosphonic acid, and their derivatives); botanical or animal-derived extracts (such as extracts or fermented products of Angelica keiskei, aloe, Fructus Rosae Multiflorae, Scutellaria root, Phellodendron bark, seaweed, Chinese quince, chamomile, licorice, kiwi, cucumber, mulberry, birch, Japanese angelica root, garlic, peony, hop, horse chestnut, lavender, rosemary, eucalyptus, milk, various peptides, placenta, royal jelly, Euglena extract, hydrolyzed Euglena extract, and Euglena oil); pH adjusters (such as inorganic acids, inorganic acid salts, organic acids, organic acid salts, and their derivatives); vitamins (including vitamin A group, vitamin B group, vitamin C, vitamin D group, ubiquinone, nicotinamide, and their derivatives); fermentation products of yeast, koji mold, and lactic acid bacteria; Galactomyces culture filtrate; whitening agents (such as tranexamic acid, cetyl tranexamic acid hydrochloride, 4-n-butylresorcinol, arbutin, kojic acid, ellagic acid, licorice flavonoids, niacinamide, and vitamin C derivatives); ceramides and ceramide derivatives; anti-wrinkle agents (such as retinol, retinal, and their derivatives, nicotinamide, oligopeptides, and their derivatives, and natural or synthetic compounds exhibiting neutrophil elastase inhibitory activity or MMP-1 and MMP-2 inhibitory activity); and powders such as titanium oxide, talc, mica, silica, zinc oxide, iron oxide, silicon, and processed forms thereof.

It should be noted that the components which may be added to the pharmaceutical and/or cosmetic composition containing the secretion from the stem cells or from the somatic cells into which the reprogramming factors have been introduced are not limited to those listed above, and any component that can be used in a pharmaceutical or cosmetic composition may be freely selected. When the pharmaceutical and/or cosmetic composition containing the secretion from the stem cells or from the somatic cells into which the reprogramming factors have been introduced according to the embodiment is used as a cataplasm (plaster), the composition may additionally contain, within a range not impairing the intended effect, a base (such as kaolin or bentonite) and a gelling agent (such as polyacrylic acid salts or polyvinyl alcohol). When the composition is used as a bath additive, it may be appropriately formulated, within the scope of achieving the intended purpose, with ingredients such as sulfates, bicarbonates, borates, dyes, and humectants, and may be prepared in the form of a powder or liquid. The pharmaceutical and/or cosmetic composition containing the secretion from the stem cells or from the somatic cells into which the reprogramming factors have been introduced according to the embodiment may also be a topical skin composition.

The pharmaceutical and/or cosmetic composition according to the embodiment may be in the form of an injectable formulation or an infusion formulation.

### EXAMPLES

### Example 1: Preparation of Supernatant From Culture Medium Used for Maintenance Culture of Stem Cells

Induced pluripotent stem (iPS) cells derived from blood cells were established by two-dimensional culture. The iPS cells were passaged more than five times, and it was confirmed that the reprogramming factors no longer remained in the iPS cells and that the iPS cells were free from viral contamination. Thereafter, the iPS cells were cryopreserved using a cryopreservation solution (STEM-CELLBANKER).

A stem cell culture medium was prepared using a medium (Puel, I Peace, Inc.) containing 1% antibiotics (Gibco Antibiotic-Antimycotic (100X), registered trademark, Thermo Fisher) and free from animal-derived components such as bFGF. A 30 mL bioreactor was prepared. Into the bioreactor, 20 mL of the stem cell culture medium supplemented with 10 μM ROCK inhibitor was added. A total of 4×10⁶ iPS cells, cryopreserved in STEM-CELLBANKER solution and including single, undifferentiated cells, were seeded into the stem cell culture medium in the bioreactor, and three-dimensional stirred culture of the iPS cells was initiated. After clump formation, the culture medium was stirred at a speed at which 20% to 60% of the clumps would sediment.

Two days later, 10 mL of stem cell culture medium was added to the bioreactor. Thereafter, stem cell culture medium was added to the bioreactor every two days until formation of cell aggregates. Flow cytometric analysis of the iPS cells on day 7 after seeding into the bioreactor showed positive expression of TRA-1-60, a pluripotency marker.

Next, the culture medium containing suspended iPS cells was filtered through a 0.22 um mesh filter to remove the cell aggregates. The filtered culture medium was then centrifuged at 1,500 rpm for 5 minutes to sediment the cells, and the supernatant of the centrifuged culture medium was collected and centrifuged again at 3,000 rpm for 3 minutes. The resulting supernatant was filtered through a 0.22 μm filter. The filtered supernatant was cryopreserved and used as the supernatant solution according to Example 1. It was confirmed that the supernatant solution was free of endotoxins and bacteria.

### Example 2: Preparation of Composition

Liposomes were prepared using hydrogenated lecithin, isopentyldiol, glycerin, soybean sterol, and liquid hydrocarbons. The prepared liposomes were suspended in the supernatant solution obtained in Example 1 to yield the composition according to Example 2.

### Example 3: Evaluation of Cosmetic Effects

The stratum corneum moisture content and the viscoelasticity of the skin to which the composition of Example 2 was applied were evaluated in order to examine the effects of the composition of Example 2 on skin hydration and firmness/elasticity. The subjects were 15 Japanese female individuals aged 20 to 59 years who self-reported dry skin. The supernatant solution of Example 1 was prepared using iPS cells derived from human donors different from the subjects. The composition of Example 2 was applied to the flexor aspect of the forearm of each subject, and measurements of skin firmness, elasticity, and stratum corneum moisture content were taken prior to application, 30 minutes after application, 1 hour after application, and 2 hours after application. As a control, the same measurements were also performed on the opposite forearm of each subject, to which the composition had not been applied.

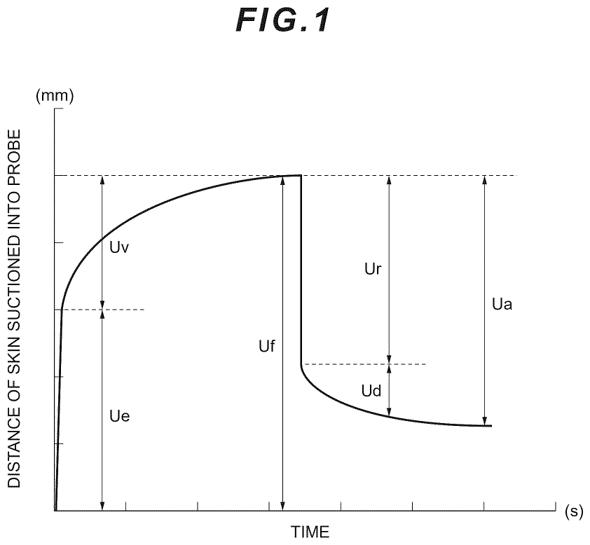

For the measurement of skin firmness and elasticity, a Cutometer® (registered trademark, Courage+Khazaka Electronic GmbH) was used. As illustrated in FIG. 1, the Cutometer adheres a probe to the skin surface and instantaneously generates negative pressure inside the probe to suction the skin into the probe. After maintaining the suction state for several seconds, the Cutometer returns the internal pressure of the probe to atmospheric pressure and releases the skin. In general, the skin does not immediately return to its original position but remains partially within the probe and gradually returns to its initial state. It is known that the suction distance (Uf), defined as the maximum displacement of the skin into the probe during suction, correlates with the flexibility of the skin. Further, the parameter Ur, which denotes the distance the skin recovers immediately after the release of suction, and the ratio of Ur to Uf (Ur/Uf), which is referred to as the elasticity ratio, are known to correlate with the elasticity of the skin, as well as with skin firmness and sagging. The measurement of stratum corneum moisture content was performed using a Corneometer® (registered trademark, Courage+Khazaka Electronic GmbH).

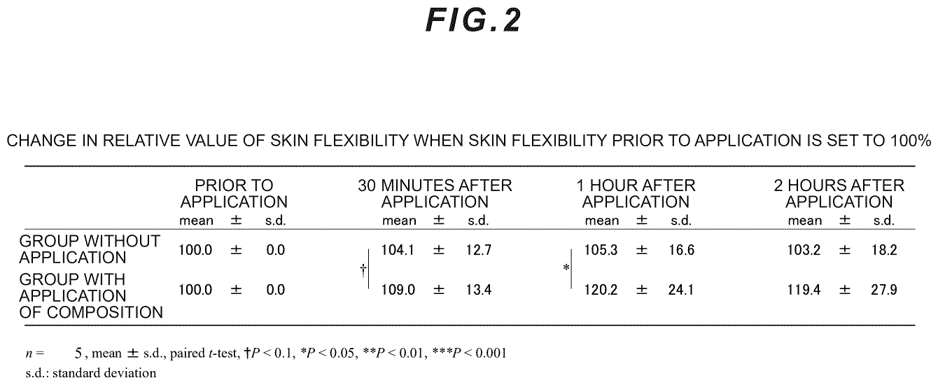

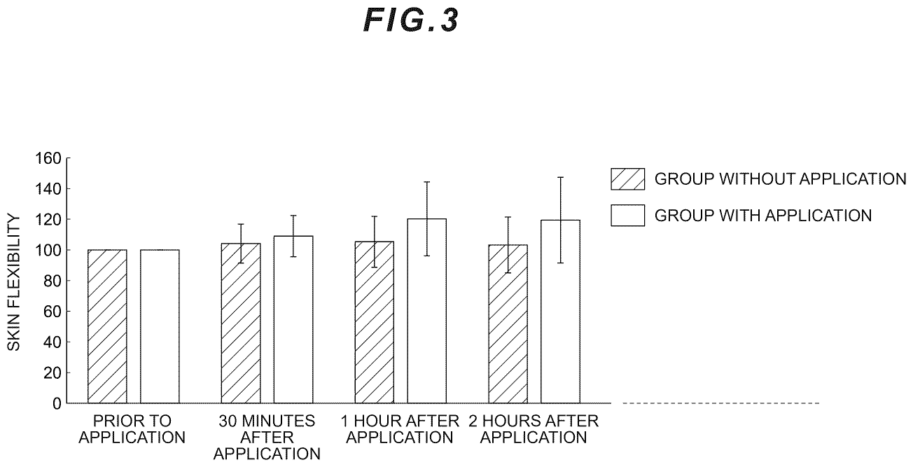

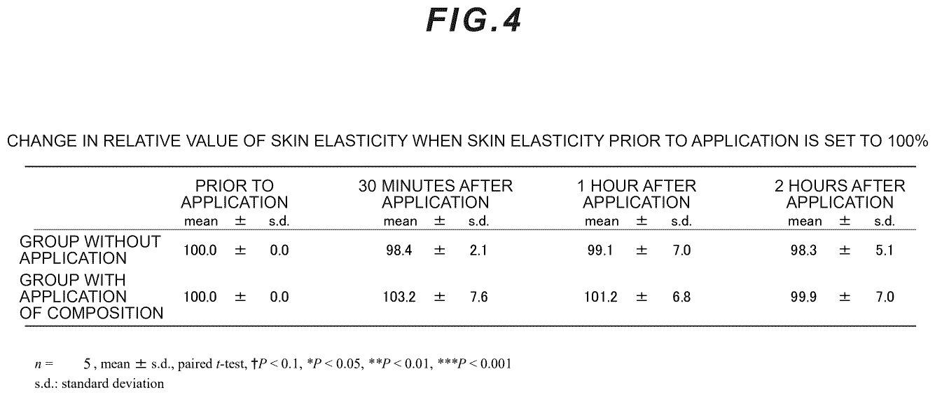

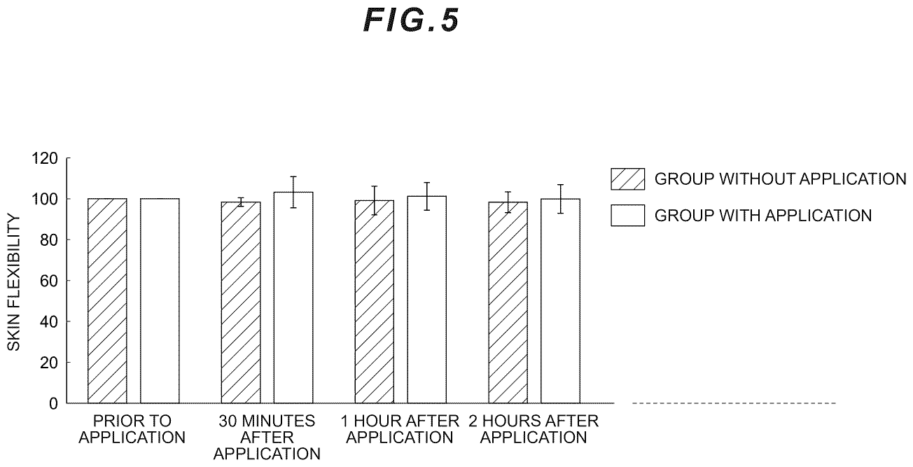

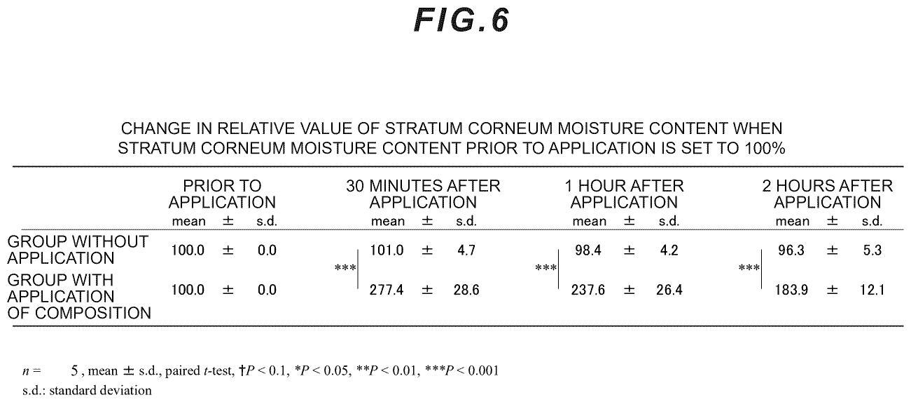

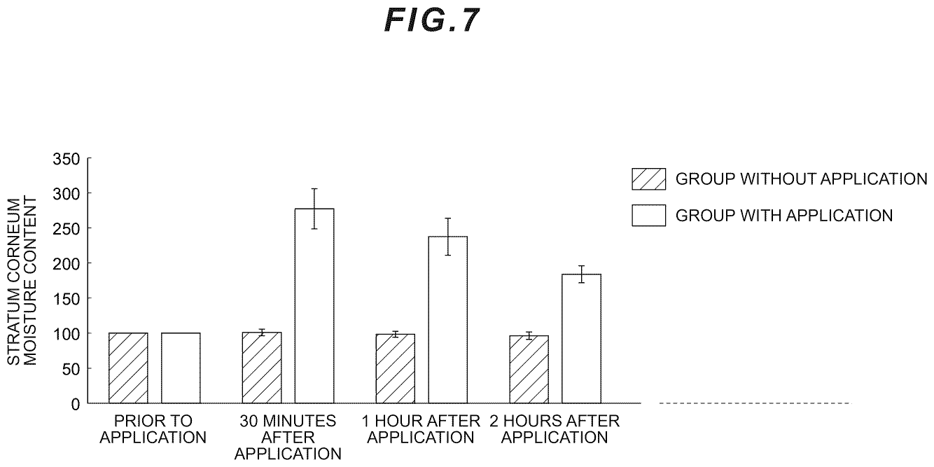

As shown in FIGS. 2 and 3, the arm to which the composition according to Example 2 was applied exhibited improved skin flexibility in comparison with the untreated arm. As shown in FIGS. 4 and 5, the arm to which the composition according to Example 2 was applied exhibited improved skin elasticity in comparison with the untreated arm. As shown in FIGS. 6 and 7, the arm to which the composition according to Example 2 was applied exhibited improved stratum corneum moisture content in comparison with the untreated arm.

### Example 4: Evaluation of Cosmetic Effects)

A supernatant solution of a culture medium in which iPS cells derived from each of 15 subjects had been cultured was prepared in the same manner as in Example 1. A composition was prepared by suspending liposomes in the supernatant solution in the same manner as in Example 2. The composition containing the supernatant solution of the culture medium of autologous iPS cells and the composition containing the supernatant solution of the culture medium of allogeneic iPS cells were applied daily for eight weeks to the volar forearm of each of the 15 subjects. Thereafter, skin elasticity and firmness were measured using a Cutometer® (registered trademark, Integral), stratum corneum moisture content was measured using a Corneometer® (registered trademark, Integral), and brown spots on the skin were measured using a Visia® Evolution (registered trademark, Integral).

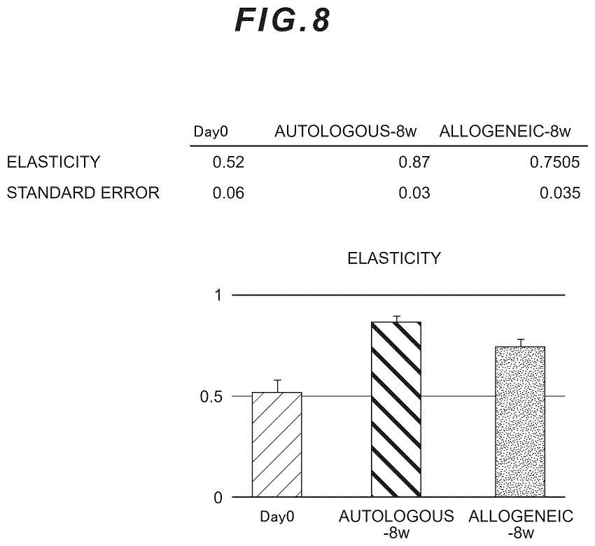

As shown in FIG. 8, both the composition containing the supernatant of the culture medium in which the autologous iPS cells were cultured and the composition containing the supernatant of the culture medium in which the allogeneic iPS cells were cultured improved skin elasticity. Furthermore, the composition containing the supernatant of the culture medium in which the autologous iPS cells were cultured exhibited a greater improvement in skin elasticity than the composition containing the supernatant of the culture medium in which the allogeneic iPS cells were cultured.

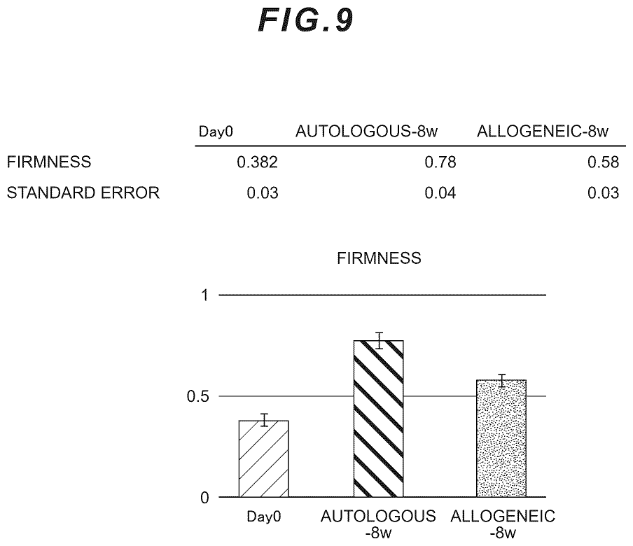

As shown in FIG. 9, both the composition containing the supernatant of the culture medium in which the autologous iPS cells were cultured and the composition containing the supernatant of the culture medium in which the allogeneic iPS cells were cultured improved skin firmness. Furthermore, the composition containing the supernatant of the culture medium in which the autologous iPS cells were cultured exhibited a greater improvement in skin firmness than the composition containing the supernatant of the culture medium in which the allogeneic iPS cells were cultured.

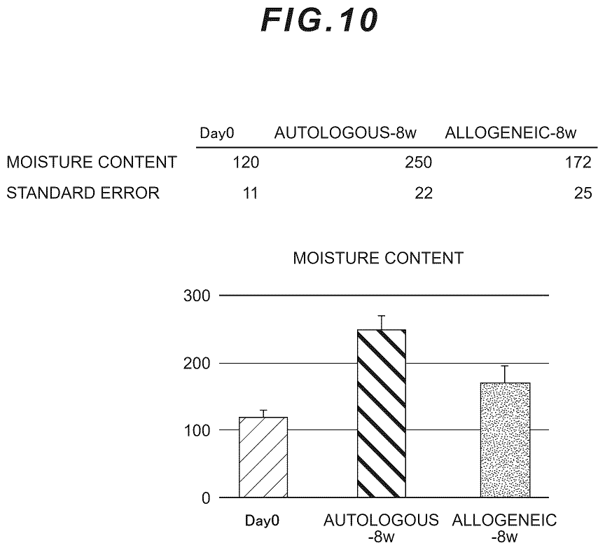

As shown in FIG. 10, both the composition containing the supernatant of the culture medium in which the autologous iPS cells were cultured and the composition containing the supernatant of the culture medium in which the allogeneic iPS cells were cultured increased the moisture content of the stratum corneum. Furthermore, the composition containing the supernatant of the culture medium in which the autologous iPS cells were cultured exhibited a greater increase in the moisture content of the stratum corneum than the composition containing the supernatant of the culture medium in which the allogeneic iPS cells were cultured.

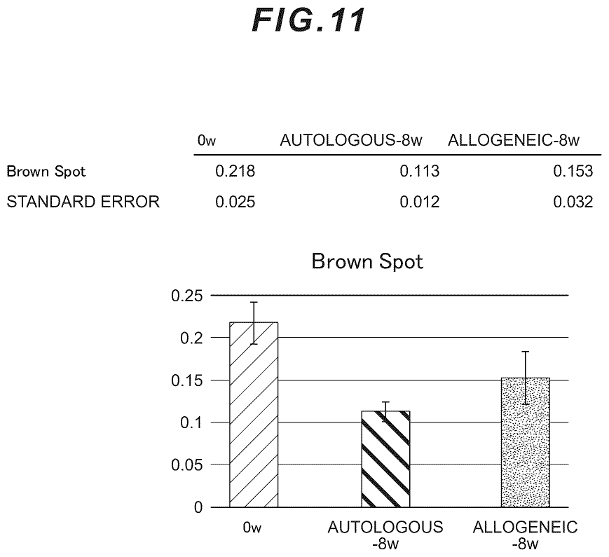

As shown in FIG. 11, both the composition containing the supernatant of the culture medium in which the autologous iPS cells were cultured and the composition containing the supernatant of the culture medium in which the allogeneic iPS cells were cultured reduced the appearance of skin spots. Furthermore, the composition containing the supernatant of the culture medium in which the autologous iPS cells were cultured exhibited a greater reduction in skin spots than the composition containing the supernatant of the culture medium in which the allogeneic iPS cells were cultured.

### Example 5: Effect of Liposomal Formulation

A cosmetic composition containing liposomes encapsulating the supernatant solution according to Example 5 was prepared by diluting the supernatant solution obtained in Example 1 to 20%, and using the diluted supernatant solution, hydrogenated lecithin, isopentyldiol, glycerin, soybean sterol, and liquid hydrocarbons. Additionally, a comparative cosmetic composition (Comparative Example of Example 5) not subjected to liposomal formulation was prepared by mixing the diluted (20%) supernatant solution with glycerin, butylene glycol (BG), dipropylene glycol (DPG), polyethylene glycol (PEG), squalane, petrolatum, amphoteric surfactant, ester oils, and unsaturated fatty acids.

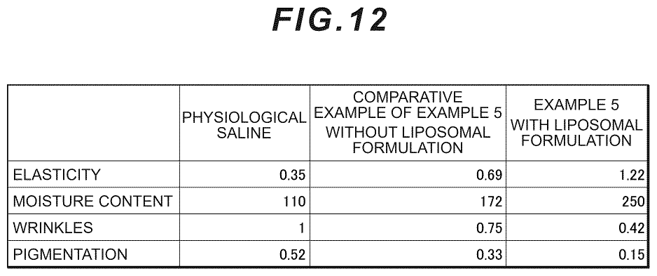

In the same manner as in Example 4, subjects were treated with physiological saline, the cosmetic composition according to Example 5, and the cosmetic composition of the comparative example of Example 5. Skin elasticity, moisture content, and pigmentation (spots) were evaluated. In addition, wrinkles were measured using VISIA Evolution™ (Integral). As shown in FIG. 12, both the cosmetic composition according to Example 5 and the comparative example of Example 5 improved skin elasticity and moisture content and reduced wrinkles and pigmentation compared to physiological saline. Furthermore, the cosmetic composition according to Example 5, which included the supernatant solution in liposomal form, showed greater improvements in skin elasticity and moisture content and greater reductions in wrinkles and pigmentation than the comparative example of Example 5, which did not include liposomal formulation.

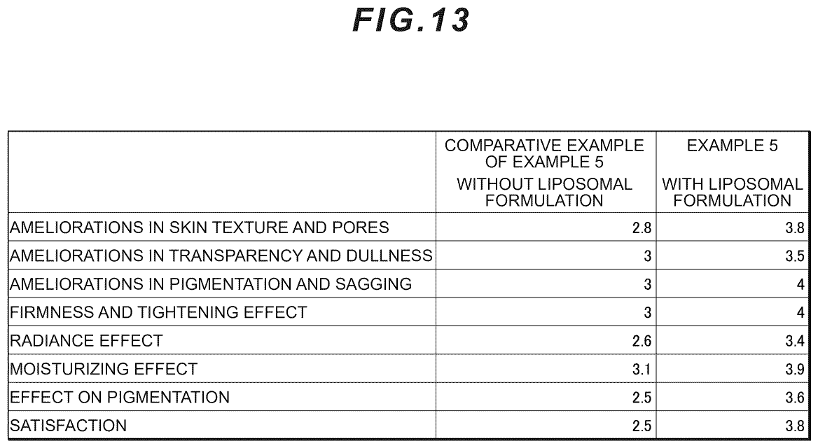

Further, a questionnaire was administered to five subjects regarding ameliorations in skin texture and pores, transparency and dullness, pigmentation and sagging, firmness and tightening effect, radiance effect, moisturizing effect, effect on pigmentation, and overall satisfaction. As shown in FIG. 13, the cosmetic composition of Example 5 that contained liposomal-formulated supernatant solution received higher evaluations than the cosmetic composition of the comparative example of Example 5 that did not contain liposomal formulation.

### Reference Example 1: Preparation of Supernatant of Culture Medium Used for Maintenance Culture of Stem Cells

A culture medium having a total volume of 500 mL was prepared by mixing 5 mL of L-glutamine (25030-081, Invitrogen), 5 mL of non-essential amino acid solution (11140-050, Invitrogen), 1 mL of 2-mercaptoethanol (21985-023, Invitrogen), and 2.5 mL of penicillin/streptomycin (15140-122, Invitrogen) with DMEM/F12 (10565-018, Invitrogen) and a serum substitute (KSR: KnockOut Serum Replacement, registered trademark, 10828028, Invitrogen). To this medium, 0.2 mL of 10 μg/mL bFGF (basic fibroblast growth factor, R&D) was added to prepare a stem cell culture medium. The concentration of bFGF in the stem cell culture medium was 4 ng/mL.

Using the stem cell culture medium prepared as described above, human iPS cells were maintained in an adherent culture on feeder cells in a culture dish for adherent culture. The human iPS cells were passaged weekly. During passaging, the human iPS cells were treated with a dissociation solution containing 0.25% trypsin, 0.1 mg/mL collagenase IV, 1 mmol/L CaCl₂, and 20% KSR.

The maintained human iPS cells were detached from the culture dish using an ES cell dissociation solution (TrypLE Select, registered trademark, Thermo Fisher). The detached human iPS cells were seeded onto a dish coated with laminin (Nippi). Thereafter, the human iPS cells were cultured for one week in Puel medium (I Peace, Inc.) supplemented with 10 μmol/L ROCK inhibitor. The culture medium was replaced daily.

Two days later, the supernatant of the Puel medium was collected. The collected supernatant of the stem cell culture medium was centrifuged at 1500 rpm for 5 minutes to remove any remaining cells. The resulting supernatant was again centrifuged at 3000 rpm for 3 minutes, and then filtered through a 0.22 um filter. The filtered supernatant was designated as the supernatant solution of Reference Example 1.

In addition, it was confirmed that the maintained iPS cells were positive for the undifferentiated markers NANOG, OCT3/4, and TRA 1-60.

### Reference Example 2: Preparation of Supernatant of Medium Used for Maintenance Culture of Stem Cells

Human iPS cells were maintained and cultured in the same manner as in Reference Example 1. Thereafter, the human iPS cells were detached from the adherent culture dish and dissociated into single cells, also in accordance with Reference Example 1. Next, the human iPS cells were seeded into a gelled stem cell medium, which had been gelled by the addition of gellan gum and 10 μmol/L ROCK inhibitor (Selleck), and were subjected to suspension culture for 21 days. During the culture period, the gelled stem cell medium was replenished into the culture vessel every two days.

Subsequently, the gelled stem cell medium containing the suspended human iPS cells was filtered through a mesh filter to remove the cell aggregates. The filtered gelled stem cell medium was then centrifuged at 1500 rpm for 5 minutes to precipitate the cells and gel. The supernatant of the stem cell medium after centrifugation was collected and centrifuged again at 3000 rpm for 3 minutes. The resulting supernatant of the stem cell medium was then filtered through a 0.22 μm filter. The filtered supernatant of the stem cell medium was designated as the supernatant solution according to Reference Example 2.

It was also confirmed that the maintained iPS cells were positive for NANOG, OCT3/4, and TRA-1-60, which are markers of undifferentiated state.

### Reference Example 3: Preparation of Supernatant of Medium Used for Maintaining Culture of Stem Cells

Human iPS cells were maintained in culture in the same manner as in Reference Example 1. Thereafter, the human iPS cells were detached from the adhesive culture dish and dissociated into single cells in the same manner as in Reference Example 1. Subsequently, the human iPS cells were seeded into a stem cell medium that had been gelled by the addition of gellan gum and 100 μmol/L ROCK inhibitor (Selleck), and were cultured under suspension conditions for 14 days. During this period, the gelled stem cell medium was replenished in the incubator every other day.

Thereafter, the gelled stem cell medium containing the suspended human iPS cells was filtered through a mesh filter to remove cell aggregates. The filtered gelled stem cell medium was then centrifuged at 1500 rpm for 5 minutes to precipitate the cells and gel. The supernatant of the stem cell medium obtained after centrifugation was collected again and subjected to a second centrifugation at 3000 rpm for 3 minutes. The resulting supernatant was filtered through a 0.22 μm filter. The filtrate obtained was defined as the supernatant solution according to Reference Example 3.

In addition, it was confirmed that the maintained human iPS cells were positive for the undifferentiated cell markers NANOG, OCT3/4, and TRA-1-60.

### Reference Comparative Example: Preparation of Supernatant of Medium in Which Differentiated Cells Were Cultured

Human iPS cells were cultured in accordance with the example described in Japanese Unexamined Patent Application Publication No. 2016-128396. Specifically, using the same stem cell culture medium as in Reference Example 1, human iPS cells were maintained by adherent culture on feeder cells on a culture dish for adherent culture. The human iPS cells were passaged weekly. During passaging, the human iPS cells were treated with a dissociation solution containing 0.25% trypsin, 0.1 mg/mL collagenase IV, 1 mmol/L CaCl₂, and 20% KSR.

The human iPS cells cultured as described above were detached from an adherent culture dish using an ES cell dissociation solution (TrypLE Select, trademark, Thermo Fisher). The detached human iPS cells were then subjected to suspension culture for one week in a non-adherent culture dish in a non-gelled medium for human iPS cells. As a result, embryoid bodies (EBs) were formed. The formed embryoid bodies were seeded onto an adherent culture dish and allowed to grow (outgrowth) for one week in DMEM containing 10% FBS.

Next, the cells were detached from the adherent culture dish using a 0.05% trypsin-EDTA solution, and the cells were dissociated into single cells and seeded onto a new adherent culture dish. Thereafter, the cells were cultured for one week using DMEM containing 10% FBS as the medium.

After confirming that the cells had reached 70% to 80% or more confluency, the medium was replaced with a serum-free medium (DMEM not containing FBS), and the cells were further cultured for two days. The supernatant of the medium was then collected. The collected medium supernatant was centrifuged at 1,500 rpm for 5 minutes, and the supernatant was collected again. Subsequently, the supernatant was further centrifuged at 3,000 rpm for 3 minutes, and the final collected supernatant was designated as the supernatant solution according to the reference comparative example.

Further, it was confirmed that the cultured cells were negative for the undifferentiated markers NANOG, OCT3/4, and TRA-1-60, and thus were differentiated cells.

### Reference Example 4: Proliferation Assay of Fibroblasts

Proliferation medium A was prepared by supplementing DMEM medium with 10% FBS and 1% penicillin-streptomycin. Next, normal human adult-derived fibroblasts (KF-4109, Strain No. 01035, Kurabo) were suspended in proliferation medium A at a concentration of 5×10³ cells/ 0.1 mL/well and seeded into 96-well plates. The cells were cultured in a CO₂ incubator (5% CO₂, 37 °C) for one day.

Test medium A was prepared by supplementing DMEM medium with 1% FBS and 1% penicillin-streptomycin. Next, each of the supernatant solutions obtained in Reference Examples 1 to 3 and the Reference Comparative Example was mixed with test medium A at a volume ratio of 10.00:90.00 to prepare supernatant-supplemented medium A containing the supernatants of Reference Examples 1 to 3 and the Reference Comparative Example at a concentration of 10.00 v/v %. The proliferation medium A in some wells was replaced with each of the supernatant-supplemented media A derived from Reference Examples 1 to 3 and the Reference Comparative Example.

As a negative control, the proliferation medium A in certain wells was replaced with DMEM medium (non-supplemented test medium A) that was not supplemented with 1% FBS or 1% penicillin-streptomycin. Additionally, as another negative control, diluted test medium A was prepared by mixing DMEM/F12 and test medium A at a volume ratio of 10.00:90.00, and the proliferation medium A in some wells was replaced with this diluted test medium A.

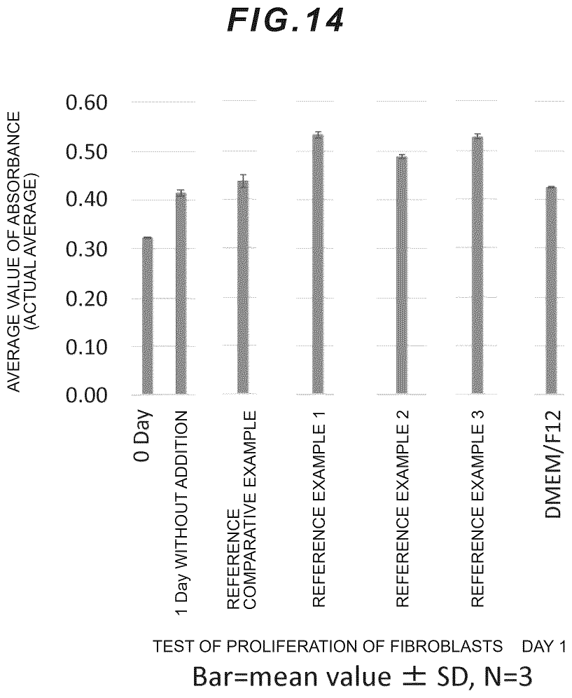

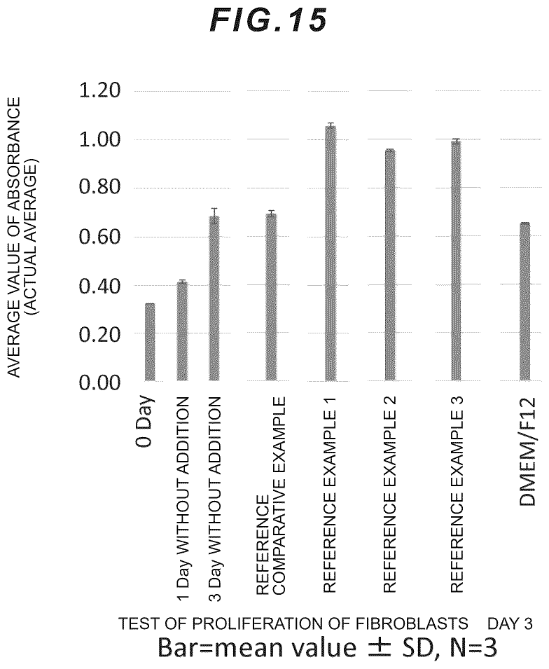

The fibroblasts were cultured for one day and three days using the replaced medium. The number of viable cells was measured by the WST-8 method using a viable cell assay reagent SF (Cat. No. 07553-15, Nacalai Tesque) and a microplate reader (Varioskan MicroPlate Reader, Thermo Scientific). The results are shown in FIGS. 14 and 15. When the supernatant-supplemented medium A containing 10.00 v/v % of the supernatants according to Reference Examples 1 to 3 was used, it was confirmed that fibroblasts proliferated significantly compared to the unsupplemented test medium A, the diluted test medium A, and the supernatant-supplemented medium A according to the Reference Comparative Example. These results suggest that the supernatants of the stem cell culture medium are effective in enhancing cell viability. Furthermore, during cell passage, when cells are detached from the culture dish or dissociated into single cells, they are subjected to physical stress such as pressure and chemical stress due to the detachment agents. However, since the cells that experienced such stress proliferated in the supernatant-supplemented medium according to the Reference Examples, it is suggested that the supernatant-supplemented medium according to the Reference Examples alleviates the stress on cells, protects the cells from stress, stabilizes the cells, and enhances their viability. These findings suggest that the supernatant-supplemented medium according to the Reference Examples protects nucleic acids, proteins, protein complexes, lipoproteins, ribosomes, and biomembranes contained in the cells. Here, the term “biomembranes” includes the cell membrane.

### Reference Example 5: Type I Collagen and Hyaluronic Acid Production Assay Using Fibroblasts

In the same manner as in Reference Example 4, adult-derived normal human fibroblasts were cultured for one day in proliferation medium A. Thereafter, except that the concentration was set to 1.00 v/v %, 10.00 v/v %, or 100.0 v/v %, portions of the proliferation medium A in individual wells were replaced with each of the supernatant-added media A corresponding to Reference Examples 1 to 3 and the reference comparative example, in the same manner as in Reference Example 4.

As a positive control, the proliferation medium A in some of the wells was not replaced. As negative controls, the proliferation medium A in certain wells was replaced with additive-free test medium A, and in other wells, replaced with the diluted test medium A prepared in the same manner as in Reference Example 4.

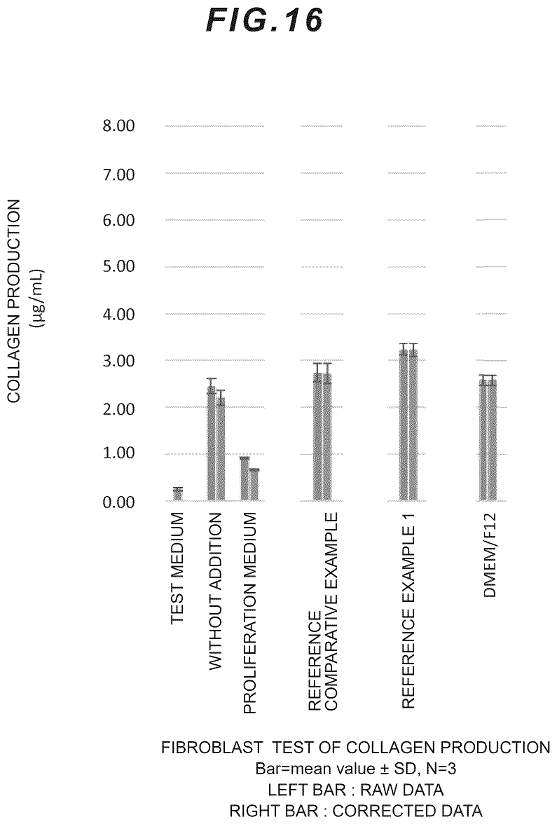

After the medium replacement, fibroblasts were cultured for three days, and the supernatant of the medium was collected and stored at −80 °C. Thereafter, the supernatant was thawed, and the concentration of type I collagen in the medium supernatant was measured using a Human Collagen Type I ELISA kit (Cat. No. EC1-E105). In addition, the concentration of hyaluronic acid in the medium supernatant was measured using the DuoSet Hyaluronan kit (Cat. No. DY3614, R&D Systems). The results are shown in FIGS. 16, 17, and 18.

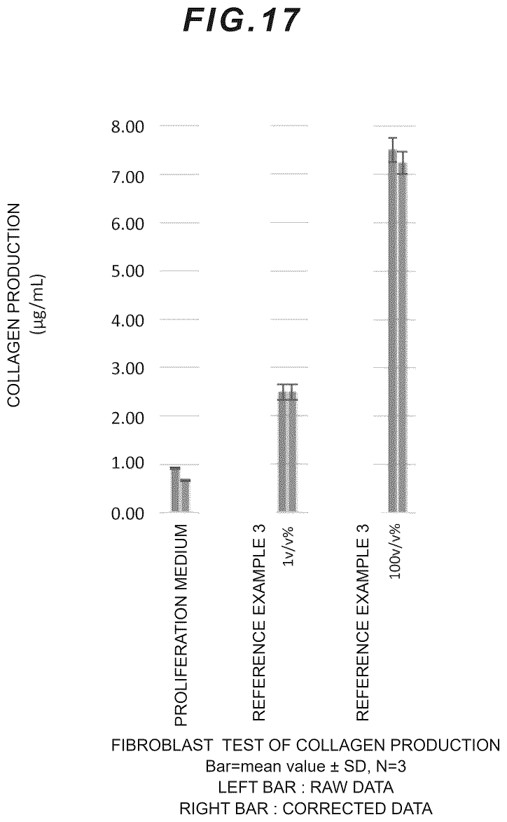

In FIGS. 16 and 17, the corrected bars on the right represent data corrected by subtracting the amount of type I collagen originally present in the culture medium before culturing the fibroblasts. The uncorrected bars on the left represent the raw data before correction. As shown in FIG. 16, when using the supernatant-supplemented medium A according to Reference Example 1 at a concentration of 1.0 v/v %, the amount of type I collagen produced was significantly increased compared to the cases using the unsupplemented test medium A, the proliferation medium A, the diluted test medium A, and the supernatant-supplemented medium A according to the Comparative Reference Example. As shown in FIG. 17, when using the supernatant-supplemented medium A according to Reference Example 3 at concentrations of 1.0 v/v % and 100.0 v/v %, the amount of type I collagen produced was markedly increased compared to the proliferation medium A. These results suggest that the supernatant of the stem cell culture medium promotes collagen production and is effective in preventing and ameliorating the formation of wrinkles and sagging skin.

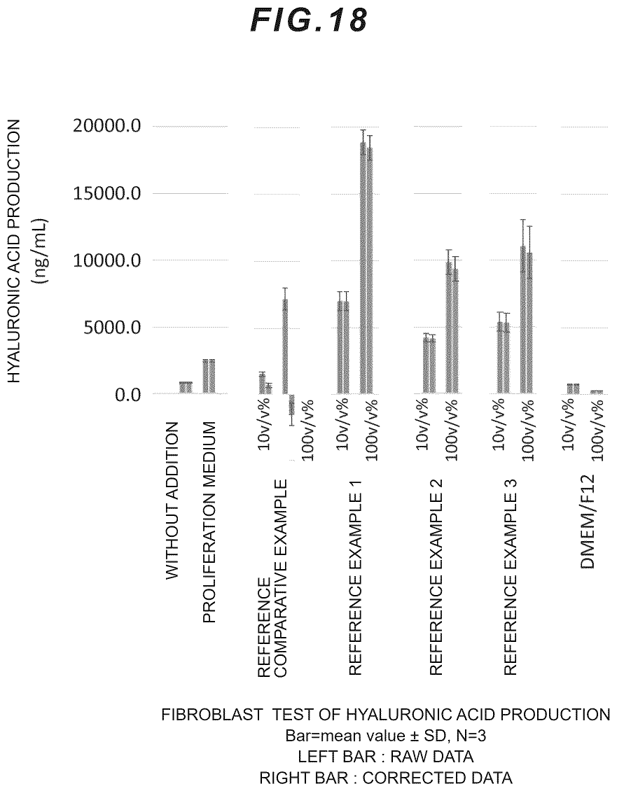

In FIG. 18, the corrected bars on the right represent data corrected by subtracting the amount of hyaluronic acid originally present in the culture medium before culturing the fibroblasts. The uncorrected bars on the left represent the raw data before correction. When using the supernatant-supplemented medium A according to Reference Examples 1 to 3 at concentrations of 10.0 v/v % and 100.0 v/v %, the amount of hyaluronic acid produced was significantly increased compared to the unsupplemented test medium A, the diluted test medium A, and the supernatant-supplemented medium A according to the Comparative Reference Example. These results suggest that the supernatant of the stem cell culture medium promotes hyaluronic acid production and is effective in preventing and ameliorating the formation of wrinkles and sagging caused by a reduction in hyaluronic acid.

### Reference Example 6: Evaluation of Epidermal Cell Migration Ability

As the proliferation medium B, 500 mL of epidermal cell culture medium (HuMedia-KG2, Kurabo) containing proliferation supplements (10 μg/mL insulin, 0.1 ng/ml hEGF, 0.67 μg/mL hydrocortisone, and 4 μL/mL bovine pituitary extract (BPE)) and antimicrobials (50 μg/mL gentamicin and 50 ng/mL amphotericin) was prepared.

Normal human epidermal cells derived from an adult were treated with 10 μg/mL of Mitomycin C (Cat. No. 20898-21, Nacalai Tesque) for 2 hours to inhibit cell division. Subsequently, the human epidermal cells were suspended in the proliferation medium B at a concentration of 4×10⁴ cells/ 0.1 mL/well and seeded into a collagen-coated plate of a cell migration assay kit (Oris Cell Migration Assay, registered trademark) to evaluate the migration ability of the cells. The cells were cultured for one day in a CO₂ incubator (5% CO₂, 37 °C), and were allowed to adhere to the periphery outside the stoppers on the plate. Thereafter, the stoppers were removed from the plate.

As the test medium B, a medium was prepared by adding antimicrobials (50 g/mL gentamicin and 50 ng/mL amphotericin) to 500 mL of epidermal cell culture medium. Subsequently, each of the supernatant solutions according to Reference Examples 1 to 3 and the Reference Comparative Example was mixed with the test medium B at volume ratios of 10.0:90.0 and 1.0:99.0, thereby obtaining supernatant-supplemented media B corresponding to Reference Examples 1 to 3 and the Reference Comparative Example. The proliferation medium B in some wells of the plate was replaced with the respective supernatant-supplemented media B.

As a negative control, the proliferation medium B in some of the wells was replaced with an epidermal cell culture medium not supplemented with proliferation additives (unsupplemented test medium B). Additionally, as another negative control, a diluted test medium B was prepared by mixing DMEM/F12 and the test medium B at volume ratios of 10.0:90.0 and 1.0:99.0, and the proliferation medium B in some wells was replaced with the resulting diluted test medium B.

During the wound healing process, epidermal cells migrate toward the wound and the wound contracts. In the present reference example, whether or not epidermal cells migrated into the area that had been covered by the stopper was analyzed using a plate reader. Specifically, 23 hours after the medium replacement, the epidermal cells were stained with a live-cell staining reagent (Calcein A M, Cat. No. 341-07901, Dojindo), and fluorescence at a wavelength of 538 nm in response to excitation light at a wavelength of 485 nm was measured using a plate reader (Varioskan Microplate Reader, Thermo Scientific).

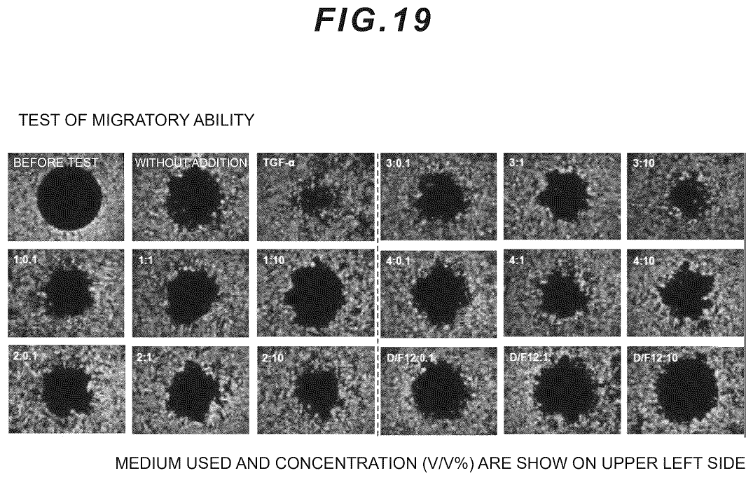

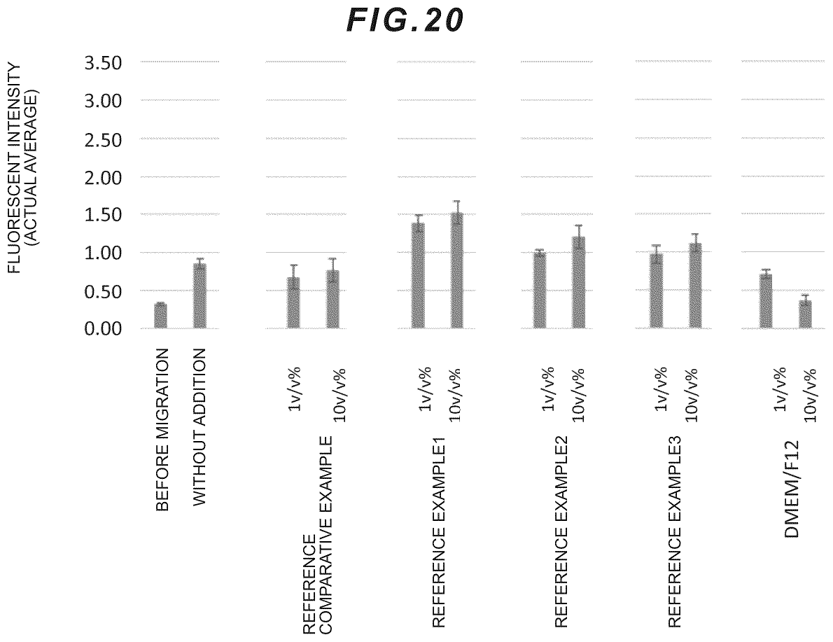

The results are shown in FIGS. 19 and 20. When the supernatant-supplemented medium B according to Reference Examples 1 to 3 was used at concentrations of 1.0 v/v % and 10.0 v/v %, a significant enhancement in the migratory ability of epidermal cells was observed, compared to the unsupplemented test medium B, the diluted test medium B, and the supernatant-supplemented medium B according to the Reference Comparative Example. Accordingly, it was demonstrated that the supernatant of a medium used for culturing stem cells is effective for wound healing. Furthermore, it was suggested that the supernatant of the stem cell culture medium is effective for preventing and ameliorating pigmentation and uneven skin tone caused by ultraviolet-induced skin damage. It was also suggested that the supernatant of the stem cell culture medium stabilizes cells and is effective in enhancing cell viability. Moreover, during passaging or dissociation into single cells, cells are subjected to physical stress, such as shear forces, and chemical stress, such as exposure to dissociation reagents. However, since cells exposed to such stress proliferated in the supernatant-supplemented media of the Reference Examples, it was suggested that the supernatant-supplemented media according to the Reference Examples alleviated cellular stress, protected the cells from stress, stabilized the cells, and improved viability. These results suggest that the supernatant-supplemented media according to the Reference Examples protect nucleic acids, proteins, protein complexes, lipoproteins, ribosomes, and biomembranes contained in cells. Here, the term “biomembrane” includes the cell membrane.

### Reference Example 7: Proliferation Assay of Hair Papilla Cells

As the proliferation medium C, a dedicated medium for hair papilla cells supplemented with specialized additives (fetal bovine serum, insulin-transferrin-triiodothyronine mixed solution, bovine pituitary extract, cyproterone acetate) (Cat. No. TMTPGM-250, TOYOBO) was prepared. Subsequently, normal human hair papilla cells (Cat. No. CA 60205a, Lot. No. 2868, TOYOBO) were suspended in the proliferation medium C at a concentration of 1.2×10⁴ cells/ 0.3 mL/well and seeded in type I collagen-coated 48-well plates. The cells were cultured for one day in a CO₂ incubator (5% CO₂, 37 °C).

Each of the supernatant solutions according to Reference Examples 1 to 3 and the Reference Comparative Example was mixed with the hair papilla cell-specific medium without additives (unsupplemented test medium C) at a volume ratio of 30.0:70.0 to prepare supernatant-supplemented media C corresponding to Reference Examples 1 to 3 and the Reference Comparative Example. The proliferation medium C in certain wells was replaced with each of the supernatant-supplemented media C.

As a negative control, the proliferation medium C in certain wells was replaced with the hair papilla cell-specific medium without additives (unsupplemented test medium C). Additionally, as another negative control, the proliferation medium C in certain wells was replaced with a diluted test medium C, prepared by mixing DMEM/F12 and the unsupplemented test medium C at a volume ratio of 30.0:70.0.

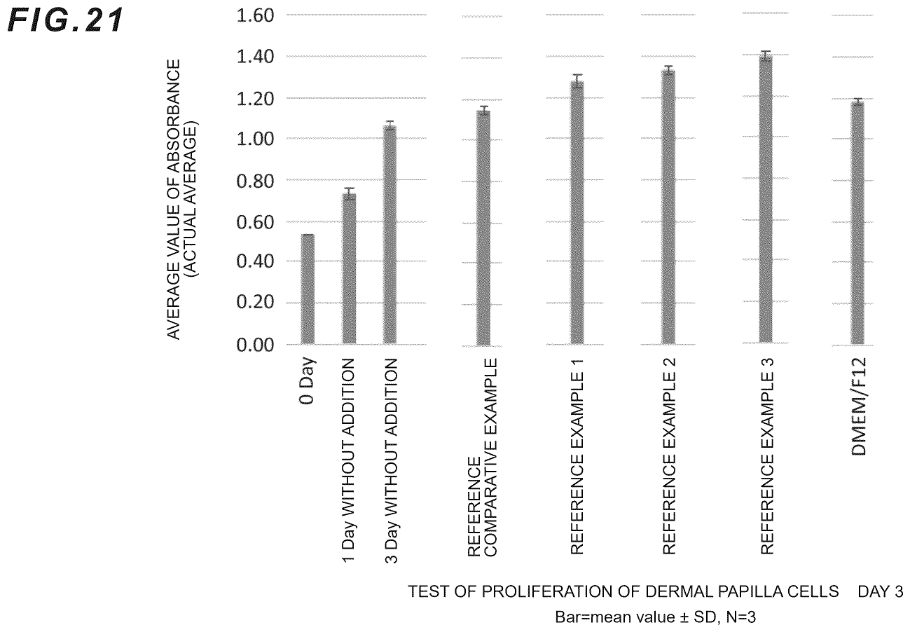

In the medium after replacement, dermal papilla cells were cultured for 3 days, and the number of viable cells was measured using the WST-8 assay. The results are shown in FIG. 21. When the supernatant-supplemented medium C according to Reference Examples 1 to 3 at a concentration of 30.0 v/v % was used, it was confirmed that the dermal papilla cells proliferated significantly compared to the cases where the non-supplemented test medium C and the diluted test medium C were used. Accordingly, it was suggested that the culture supernatant of stem cells has hair growth and hair regeneration effects such as treatment of thinning hair, prevention of hair loss, promotion of hair growth, and enhancement of hair regeneration.

### Reference Example 8: Evaluation of FGF-7 and VEGF Production by Dermal Papilla Cells

In the same manner as in Reference Example 7, normal human dermal papilla cells were cultured for 1 day in proliferation medium C. Thereafter, except for using concentrations of 0.3 v/v %, 30.0 v/v %, or 100.0 v/v %, the proliferation medium C in some of the wells was replaced with each of the supernatant-supplemented media C according to Reference Examples 1 to 3 and the comparative reference example, in the same manner as in Reference Example 7.

As negative controls, the proliferation medium C in some wells was replaced with each of the non-supplemented test medium C and the diluted test medium C. As reference controls, the proliferation medium C in some wells was replaced with an adenosine-supplemented medium prepared by adding 100 μmol/L of adenosine to the dermal papilla cell-specific medium, and with a minoxidil-supplemented medium prepared by adding 30 μmol/L of minoxidil to the dermal papilla cell-specific medium. Furthermore, as a vehicle control for minoxidil, the proliferation medium C in some wells was replaced with a DMSO-supplemented medium prepared by adding 0.1% DMSO to the dermal papilla cell-specific medium.

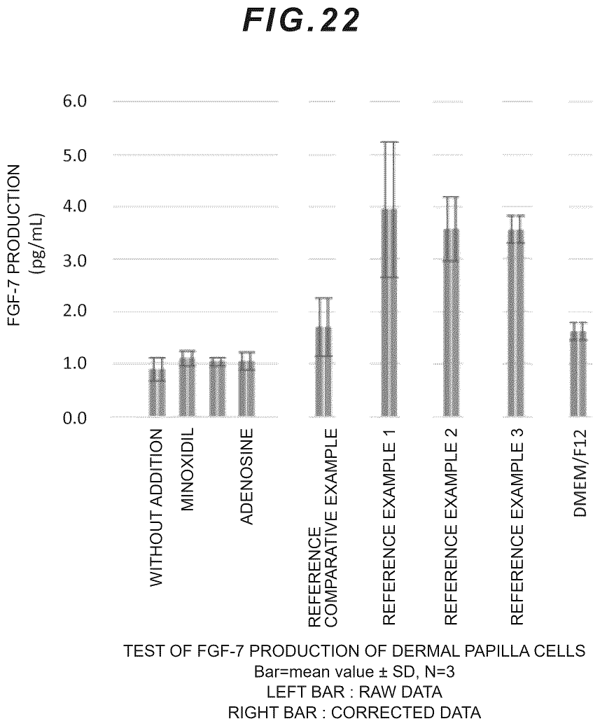

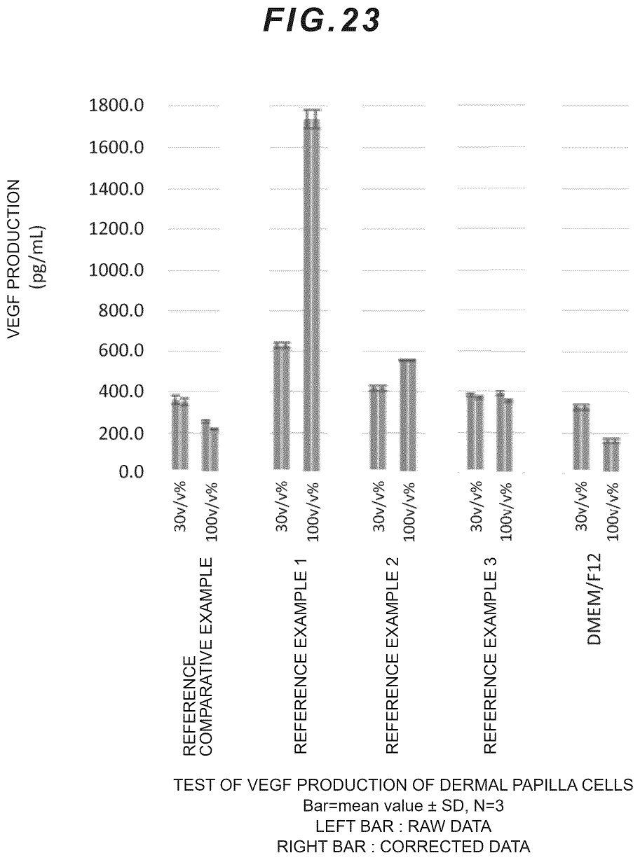

After the medium was replaced, the dermal papilla cells were cultured for 3 days, and the supernatant of the medium was collected and stored at −80 °C. Thereafter, the supernatant was thawed, and the concentration of fibroblast growth factor 7 (FGF-7) in the medium supernatant was measured using an FGF-7 Human ELISA Kit (Cat. No. ab100519, Abcam). In addition, the concentration of vascular endothelial growth factor (VEGF) in the medium supernatant was measured using the Human VEGF Quantikine ELISA Kit (Cat. No. DVE00, R&D Systems). The results are shown in FIGS. 22 and 23.

In FIG. 22, the corrected bars on the right side represent data adjusted to exclude the amount of FGF-7 originally contained in the medium before culturing the dermal papilla cells. The raw data bars on the left side represent uncorrected data. When using the supernatant-supplemented medium C according to Reference Examples 1 to 3 at a concentration of 0.3 v/v %, the amount of FGF-7 production was significantly increased compared to the cases using the non-supplemented test medium C, the diluted test medium C, and the supernatant-supplemented medium C according to the Comparative Reference Example.

In FIG. 23, the corrected bars on the right side represent data adjusted to exclude the amount of VEGF originally contained in the medium before culturing the dermal papilla cells. The raw data bars on the left side represent uncorrected data. When using the supernatant-supplemented medium C according to Reference Examples 1 to 3 at concentrations of 30.0 v/v % and 100.0 v/v %, the amount of VEGF production was significantly increased compared to the cases using the diluted test medium C and the supernatant-supplemented medium C according to the Comparative Reference Example.

### Example 6: Preparation of Test Substances

Test substances 1 to 4 shown in Table 1 were prepared. Test substance 1 contained only Dulbecco's Modified Eagle Medium (DMEM).

### Table 1

Test Substance 1 Dmem Only
Test Substance 2 Puel used for two-dimensional maintenance culture of human iPS cells
Test Substance 3 Isolated and concentrated exosomes derived from human iPS cells
Test Substance 4 Isolated and concentrated proteins derived from human iPS cells

Test Substance 2 was prepared by the following method. Human iPS cells were maintained in an adherent culture using a stem cell culture medium (Puel, I Peace, Inc.) on culture dishes coated with laminin 511. The human iPS cells were passaged once every week. During passaging, the human iPS cells were treated with an ES cell dissociation solution (TrypLE Select, trademark, Thermo Fisher Scientific).

The human iPS cells maintained as described above were detached from the adherent culture dishes using the ES cell dissociation solution (TrypLE Select, trademark, Thermo Fisher Scientific). The detached human iPS cells were seeded onto dishes coated with laminin (Nippi). Thereafter, the human iPS cells were cultured for one week in a stem cell culture medium (Puel, I Peace, Inc.) supplemented with 10 μmol/L ROCK inhibitor. The culture medium was replaced every other day.

When the cell density reached 2×10⁴ cells/cm² or more and confluency exceeded 30%, the culture medium was replaced with fresh medium. Two days thereafter, the supernatant of the stem cell culture medium was collected. The collected supernatant was centrifuged at 400×g for 30 minutes, and then filtered through a 0.22 μm filter. The filtered stem cell culture medium supernatant was designated as Test Substance 2. Furthermore, it was confirmed that the maintained iPS cells were positive for undifferentiated cell markers including alkaline phosphatase, NANOG, OCT3/4, SSEA4, SSEA5, and TRA1-60.

Test Substance 3 was prepared by the following method. The supernatant of the stem cell medium was collected in the same manner as for the preparation of Test Substance 2. The collected supernatant was centrifuged at 400×g for 30 minutes, and the resulting supernatant was filtered through a 0.22 μm filter. Thereafter, the proteins contained in the supernatant were concentrated more than fivefold by ultrafiltration using a centrifugal concentrator tube (VIVASPIN 20, Funakoshi). Next, exosomes were captured from the concentrated supernatant using the MagCapture™ Exosome Isolation Kit PS Ver.2 (Fujifilm Wako Pure Chemical Corporation), in accordance with the attached protocol, by binding the exosomes to magnetic beads and recovering the magnetic beads. The exosomes were then eluted from the magnetic beads and resuspended in fresh stem cell medium (Puel, I Peace, Inc.) to obtain Test Substance 3. It is noted that the exosomes may alternatively be recovered and purified using the Exosome Purification Kit filtration system (ExoIsolator, Dojindo Laboratories).

Test Substance 4 was prepared by the following method. The supernatant of the stem cell medium was collected in the same manner as for the preparation of Test Substance 2. Six milliliters of the supernatant were loaded onto an ultrafiltration membrane with a molecular weight cutoff of 5-10 kDa (Cytiva), and centrifuged at 200 to 400 ×g for 30 minutes. The proteins remaining on the membrane, having a molecular weight of 10 kDa or more, were collected as Test Substance 4. The proteins were concentrated more than fivefold by ultrafiltration.

### Example 7: Lipolysis Assay

Using a growth medium (PGM-2 supplemented with 10% FBS, 2 mM L-glutamine, 50 μg/mL gentamicin, and 37 ng/ml amphotericin B in PBM; Lonza, PT-8002), normal human subcutaneous preadipocytes (Lonza, PT-5020) were thawed into T-75 flasks and cultured in a CO₂ incubator (37 °C, 5% CO₂, humidified; hereinafter the same) until reaching 60% to 80% confluency. Subsequently, the normal human subcutaneous preadipocytes were detached from the T-75 flasks using 0.25% trypsin-EDTA and then centrifuged. Thereafter, the preadipocytes were seeded into a 96-well plate at a density of 10,000 cells/ 100 μL/well in growth medium and cultured in a CO₂ incubator.

On the following day, the medium was replaced with 100 μL of a differentiation medium (PGM-2 supplemented with insulin, dexamethasone, indomethacin, and isobutylmethylxanthine; Lonza, PT-8002), and the cells were cultured in a CO₂ incubator for 7 days to induce differentiation of the preadipocytes into adipocytes. Next, the medium was replaced with 100 μL of differentiation medium containing one of the Test Substances 1 through 4, or 100 μL of differentiation medium not containing any Test Substance as a control. The cells were then cultured in a CO₂ incubator for 24 hours to perform treatment of the adipocytes with the respective Test Substances 1 through 4.

Subsequently, the culture supernatant was collected, and the amount of free glycerol in the supernatant was measured using a lipolysis assay kit (Cayman, 10009381). The amount of free glycerol corresponds to the degree of lipolysis. The measurement was performed in accordance with the protocol provided with the kit. Specifically, the absorbance of the supernatant at a wavelength of 540 nm was measured using a multispectral microplate reader (Varioskan Flash, Thermo Fisher Scientific), and the relative concentration of free glycerol was calculated. A group to which no Test Substance was added was used as a control. The control group and each group to which a Test Substance was added were compared using a two-sided Dunnett's test, and a p-value of less than 0.05 was considered statistically significant.

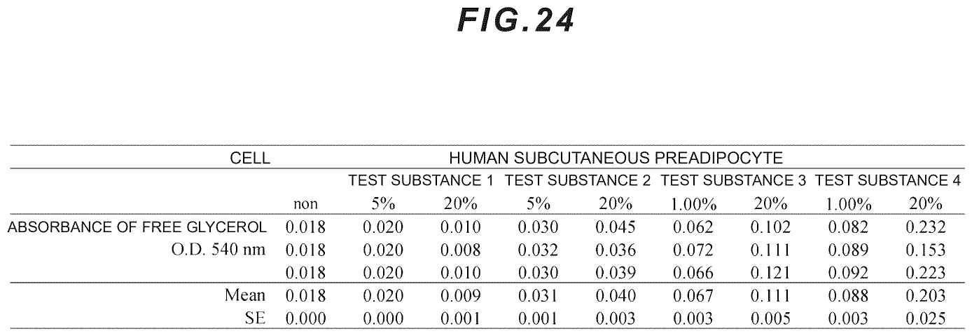

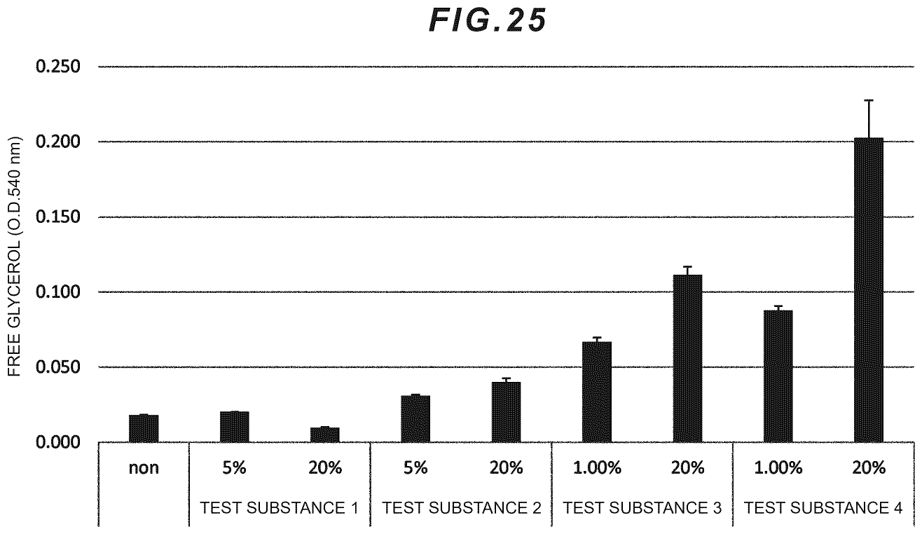

When the final concentrations of Test Substances 1 and 2 were 5%, and those of Test Substances 3 and 4 were 1%, as shown in FIGS. 24 and 25, the group to which Test Substance 1 (DMEM) was added did not exhibit a significantly higher level of lipolysis compared to the control group. In contrast, the groups to which Test Substances 2 to 4 were added exhibited significantly higher levels of lipolysis than the control group. The group to which Test Substance 3, containing the isolated and concentrated exosomes, was added exhibited significantly higher lipolysis than the group to which Test Substance 2 (culture supernatant) was added. Furthermore, the group to which Test Substance 4, containing the isolated and concentrated proteins, was added exhibited significantly higher lipolysis than the group to which Test Substance 3 was added.

Similarly, when the final concentrations of Test Substances 1 to 4 were 20%, as shown in FIGS. 24 and 25, the group to which Test Substance 1 (DMEM) was added did not exhibit significantly higher lipolysis compared to the control group. In contrast, the groups to which Test Substances 2 to 4 were added exhibited significantly higher lipolysis than the control group. The group to which Test Substance 3, containing the isolated and concentrated exosomes, was added exhibited significantly higher lipolysis than the group to which Test Substance 2 was added. Additionally, the group to which Test Substance 4, containing the isolated and concentrated proteins, was added exhibited significantly higher lipolysis than the group to which Test Substance 3 was added. Moreover, Test Substances 3 and 4 demonstrated concentration-dependent enhancement of the lipolytic effect. Notably, even at a final concentration of 1%, Test Substances 3 and 4 exhibited greater efficacy than Test Substance 2 at a final concentration of 20%.

The results of present Example demonstrate that a culture medium used for the maintenance of stem cells, exosomes isolated and concentrated from stem cells, and proteins isolated and concentrated from stem cells possess lipolytic activity.

### Example 8: Prostacyclin Production Assay

Using a culture medium (EGM-2, Lonza, CC-3162), normal human umbilical vein endothelial cells (Lonza, CC-2519AS) were seeded into T-75 flasks and cultured in a CO₂ incubator until they reached 60% to 80% confluency. Thereafter, the normal human umbilical vein endothelial cells were detached from the T-75 flasks using 0.05% trypsin-EDTA with phenol red. Subsequently, the cells were suspended in the culture medium and seeded into 96-well plates at a density of 10,000 cells/100 μL/well, followed by further culturing in the CO₂ incubator.

On the following day, the culture medium was replaced with 100 μL of medium containing one of the Test Substances 1 through 4, or, as a control, with 100 μL of medium not containing any Test Substance. The cells were then cultured for 24 hours in the CO₂ incubator.

The culture supernatant was collected, and the amount of 6-keto-prostaglandin F1α, a metabolic product of prostacyclin, was measured using an ELISA kit (Abcam, ab133023) and a multispectral microplate reader (Varioskan Flash, Thermo Fisher Scientific).

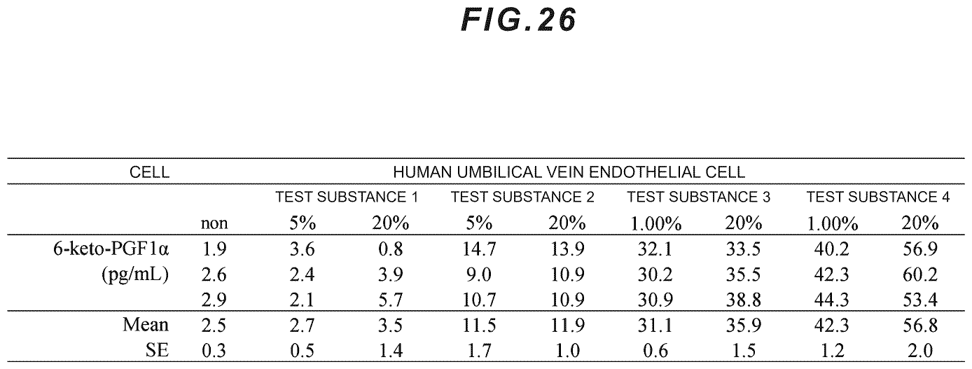

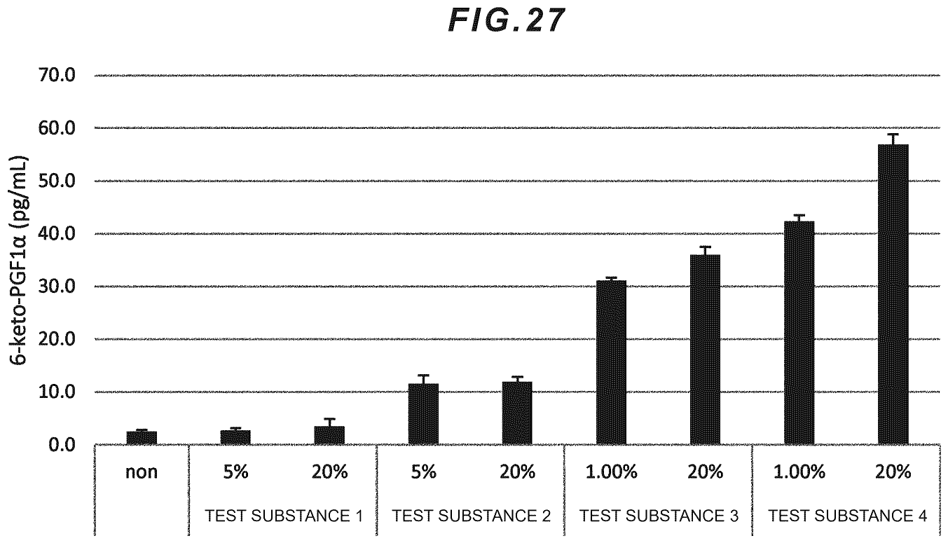

When the final concentrations of Test Substances 1 and 2 were 5%, and the final concentrations of Test Substances 3 and 4 were 1%, as shown in FIGS. 26 and 27, the group treated with Test Substance 1, which is DMEM, did not exhibit a significant increase in lipolysis compared with the control. In contrast, the groups treated with Test Substances 2 through 4 exhibited significantly higher amounts of 6-keto-prostaglandin Fla compared with the control group that was not treated with any test substance. The group treated with Test Substance 3, which contains the isolated and concentrated exosomes, showed a significantly higher amount of 6-keto-prostaglandin F1α compared with the group treated with Test Substance 2, which is the culture supernatant. Furthermore, the group treated with Test Substance 4, which contains isolated and concentrated proteins, exhibited a significantly higher amount of 6-keto-prostaglandin Fla than the group treated with Test Substance 3.

As shown in FIGS. 26 and 27, even when the final concentration of each of Test Substances 1 to 4 was 20%, the group to which Test Substance 1 (DMEM) was added did not exhibit a significant increase in the amount of lipolysis compared to the control. In contrast, the groups to which Test Substances 2 to 4 were added showed a significant increase in the amount of 6-keto-prostaglandin F1α compared to the control group to which no test substance was added. The group to which Test Substance 3, containing the isolated and concentrated exosomes, was added showed a significantly higher amount of 6-keto-prostaglandin Fla compared to the group to which Test Substance 2, containing the culture supernatant, was added. The group to which Test Substance 4, containing the isolated and concentrated proteins, was added showed a significantly higher amount of 6-keto-prostaglandin F1α compared to the group to which Test Substance 3 was added. Furthermore, Test Substances 3 and 4 exhibited dose-dependent effects. In addition, Test Substances 3 and 4, even at a final concentration of 1%, were more effective than Test Substance 2 at a final concentration of 20%.

The results of the present Example indicate that the medium used for the maintenance culture of stem cells, isolated and concentrated exosomes derived from stem cells, and isolated and concentrated proteins derived from stem cells promote the production of prostacyclin by normal human umbilical vein endothelial cells.

### Example 9: Assay for Hyaluronic Acid Production by Chondrocytes

Using a proliferation medium (DMEM/F-12 supplemented with 10% FBS and 1% penicillin-streptomycin mixed solution), human chondrocyte cell line SW1353 (ATCC, HTB-94) was thawed and seeded into a T-75 flask and cultured in a CO₂ incubator until reaching 60% to 80% confluency. Subsequently, the human chondrocytes were detached from the T-75 flask using 0.25% trypsin-EDTA with phenol red. Thereafter, using the same proliferation medium, the human chondrocytes were seeded into a 96-well plate at a density of 10,000 cells/ 100 μL/well and cultured in a CO₂ incubator.

On the following day, the medium was replaced with 100 μL of assay medium (DMEM/F-12 supplemented with 1% FBS and 1% penicillin-streptomycin mixed solution) containing each of the Test Substances 1 through 4, or with 100 μL of the assay medium not containing any Test Substance as a control. The human chondrocytes were then cultured in the CO₂ incubator for 72 hours.

The culture supernatants were collected, and the amount of hyaluronic acid in the supernatants was measured using a Hyaluronan Quantikine ELISA Kit (R&D Systems) and a multispectral microplate reader (Varioskan Flash, Thermo Fisher Scientific).

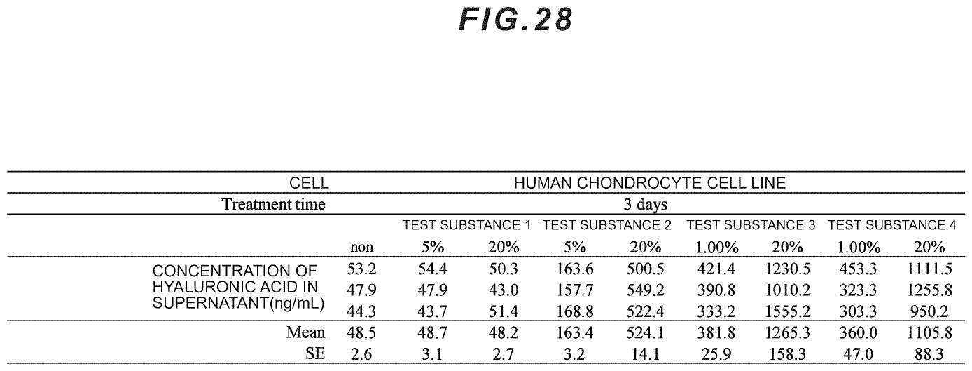

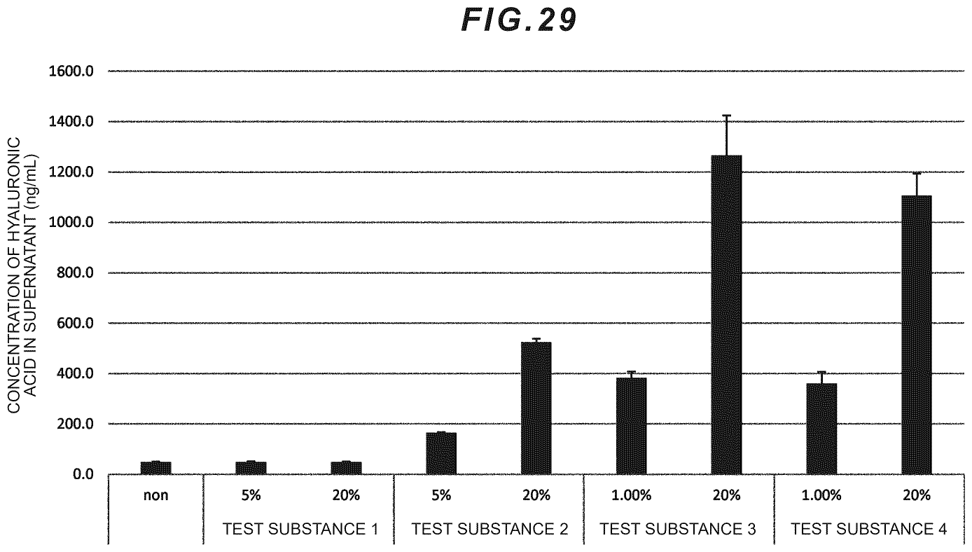

When the final concentrations of Test Substances 1 and 2 were 5%, and those of Test Substances 3 and 4 were 1%, as shown in FIGS. 28 and 29, the amount of hyaluronic acid produced in the group to which Test Substance 1, containing DMEM, was added was not significantly increased compared to the control. In contrast, in the groups to which Test Substances 2 through 4 were added, the amount of hyaluronic acid produced was significantly increased compared to the control. The group to which Test Substance 4, containing the isolated and concentrated proteins, was added showed a significantly greater production of hyaluronic acid than the group to which Test Substance 2, containing the culture supernatant, was added. Furthermore, the group to which Test Substance 3, containing the isolated and concentrated exosomes, was added showed a significantly greater production of hyaluronic acid than the group to which Test Substance 4 was added.

Similarly, when the final concentration of each of Test Substances 1 through 4 was 20%, as shown in FIGS. 28 and 29, the amount of hyaluronic acid produced in the group to which Test Substance 1, containing DMEM, was added was not significantly increased compared to the control. In contrast, in the groups to which Test Substances 2 through 4 were added, the amount of hyaluronic acid produced was significantly increased compared to the control. The group to which Test Substance 4, containing the isolated and concentrated proteins, was added showed a significantly greater production of hyaluronic acid than the group to which Test Substance 2, containing the culture supernatant, was added. The group to which Test Substance 3, containing the isolated and concentrated exosomes, was added showed a significantly greater production of hyaluronic acid than the group to which Test Substance 4 was added. In addition, Test Substances 3 and 4 exhibited concentration-dependent efficacy. Furthermore, Test Substances 3 and 4 exhibited higher effects even at a final concentration of 1% than Test Substance 2 at a final concentration of 5%.

The results of the present Example demonstrate that the culture medium used for the maintenance culture of stem cells, isolated and concentrated exosomes derived from stem cells, and isolated and concentrated proteins derived from stem cells promote the production of hyaluronic acid by human chondrocytes.

### Example 10: Test for Hyaluronic Acid Production by Fibroblasts

As growth medium A, DMEM supplemented with 10% FBS and 1% penicillin-streptomycin was prepared. Subsequently, normal adult human fibroblasts (KF-4109, Strain No. 01035, Kurabo) were suspended in the growth medium A at a concentration of 5×10³ cells/0.1 mL/well and seeded into a 96-well plate, followed by incubation in a CO₂ incubator (5% CO₂, 37 °C) for one day.

As test medium A, DMEM supplemented with 1% FBS and 1% penicillin-streptomycin was prepared. Each of the Test Substances 1 to 4 was mixed with the test medium A, and the growth medium A in some wells was replaced with the respective test medium A containing the test substances. As a control, the growth medium A in some wells was not replaced with the test medium A containing the test substances.

After the medium was replaced, the fibroblasts were cultured for three days. The culture supernatant was collected and stored at −80 °C. Subsequently, the supernatant was thawed, and the hyaluronic acid concentration in the supernatant was measured using the DueSet Hyaluronan (Cat. No. DY3614, R&D Systems).

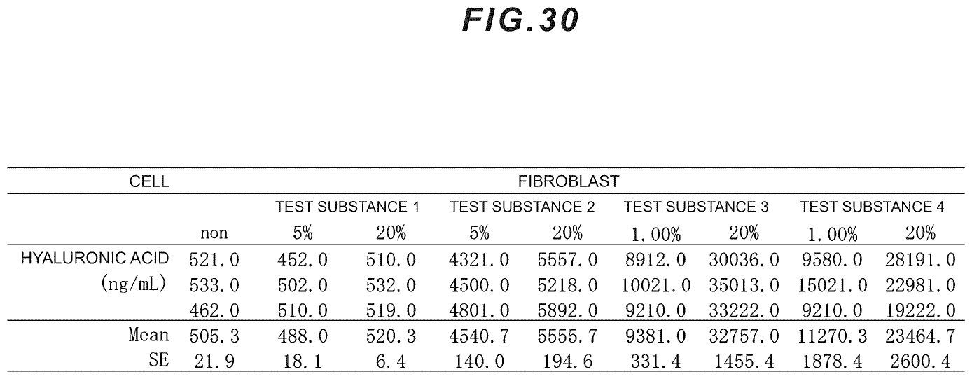

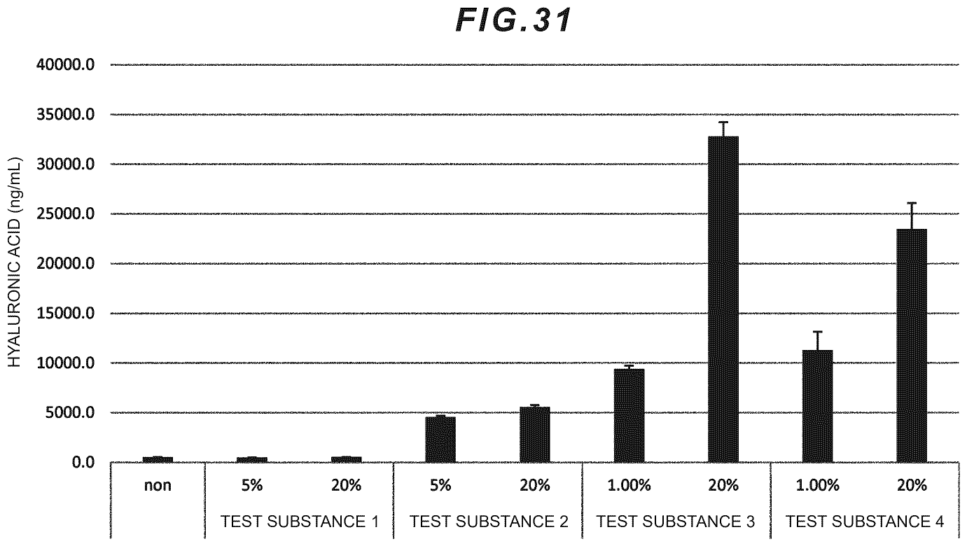

When the final concentrations of Test Substances 1 and 2 were 5%, and those of Test Substances 3 and 4 were 1%, as shown in FIGS. 30 and 31, the group to which Test Substance 1, containing DMEM, was added did not show a significant increase in hyaluronic acid production compared to the control. The groups to which Test Substances 2 to 4 were added showed significantly increased hyaluronic acid production compared to the control.

The group to which Test Substance 4, containing the isolated and concentrated proteins, was added exhibited significantly greater hyaluronic acid production compared to the group to which Test Substance 2, containing the culture supernatant, was added. The group to which Test Substance 3, containing the isolated and concentrated exosomes, was added exhibited significantly greater hyaluronic acid production compared to the group to which Test Substance 4 was added.

As shown in FIGS. 30 and 31, even when the final concentration of each of the Test Substances 1 to 4 was 20%, the group to which Test Substance 1, containing DMEM, was added did not exhibit a significant increase in the amount of hyaluronic acid production compared to the control. In contrast, the groups to which Test Substances 2 to 4 were added exhibited significantly increased production of hyaluronic acid compared to the control. The group to which Test Substance 4, containing the isolated and concentrated proteins, was added exhibited significantly increased production of hyaluronic acid compared to the group to which Test Substance 2, containing the culture supernatant, was added. The group to which Test Substance 3, containing the isolated and concentrated exosomes, was added exhibited significantly increased production of hyaluronic acid compared to the group to which Test Substance 4 was added. Moreover, Test Substances 3 and 4 exhibited a concentration-dependent increase in effect. Furthermore, Test Substances 3 and 4, even at a final concentration of 1%, exhibited a greater effect than Test Substance 2 at a final concentration of 20%.

The results of the present Example indicate that the culture medium used for the maintenance culture of stem cells, isolated and concentrated exosomes derived from stem cells, and isolated and concentrated proteins derived from stem cells promote the production of hyaluronic acid by fibroblasts.

### Example 11: Collagen Production Assay by Fibroblasts

A growth medium A was prepared, containing DMEM medium supplemented with 10% FBS and 1% penicillin-streptomycin. Subsequently, normal human fibroblasts derived from an adult (KF-4109, Strain No. 01035, Kurabo) were suspended in the growth medium A at a concentration of 5×10³ cells/ 0.1 mL/well and seeded in a 96-well plate, followed by incubation for one day in a CO₂ incubator (5% CO₂, 37 °C).

As an assay medium A, DMEM medium supplemented with 1% FBS and 1% penicillin-streptomycin was prepared. Each of the Test Substances 1 to 4 was mixed with the assay medium A, and the growth medium A in some of the wells was replaced with each of the assay media containing the respective test substances. As a control, the growth medium A in some of the wells was not replaced with the assay medium containing the test substance. Additionally, as a positive control, a mixture of ascorbic acid and the assay medium A was prepared, and the growth medium A in some of the wells was replaced with the assay medium containing ascorbic acid.

After medium replacement, fibroblasts were cultured for three days. The culture supernatant was collected and stored at −80 °C. Thereafter, the supernatant was thawed, and the concentration of type I collagen in the supernatant was measured using a human collagen type I ELISA kit (Cat. No. EC1-E105).

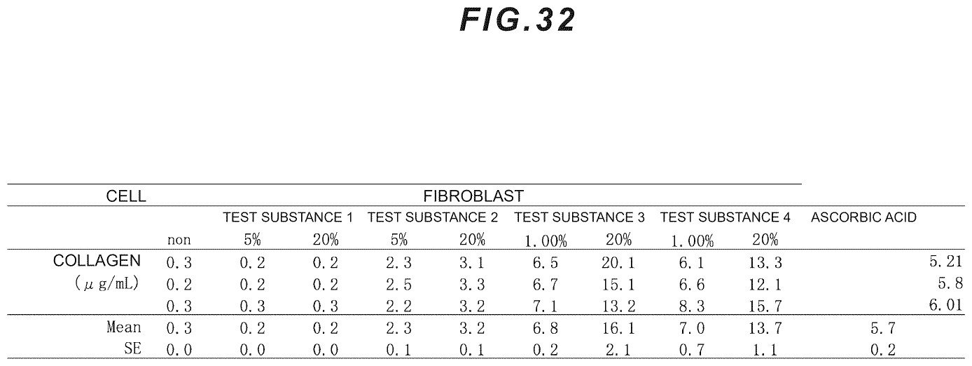

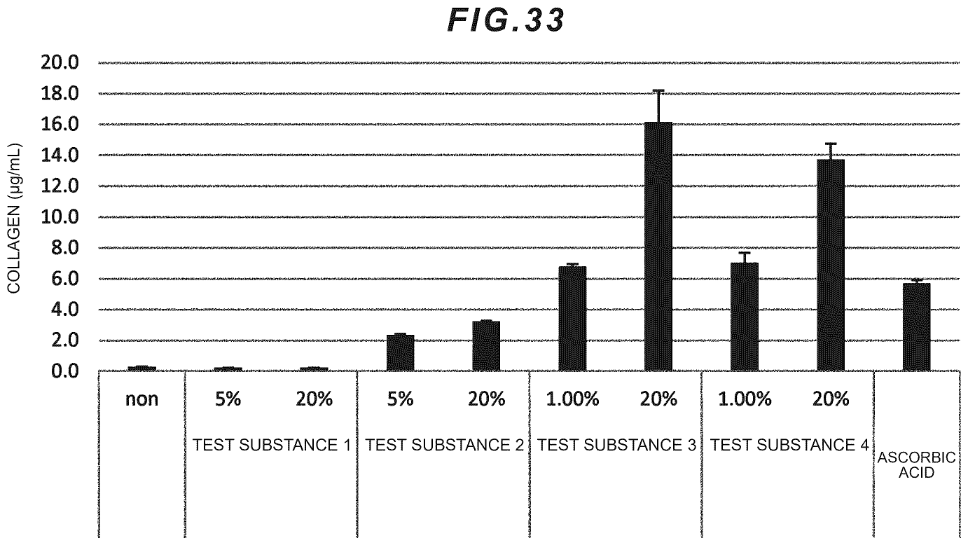

When the final concentration of Test Substances 1 and 2 was 5%, and the final concentration of Test Substances 3 and 4 was 1%, as shown in FIGS. 32 and 33, the group to which Test Substance 1 (DMEM) was added did not show a significant increase in collagen production compared to the control. In contrast, the groups to which Test Substances 2 to 4 were added exhibited significantly higher collagen production compared to the control. The group to which Test Substance 4, containing the isolated and concentrated proteins, was added showed significantly higher collagen production compared to the group to which Test Substance 2 (culture supernatant) was added. The group to which Test Substance 3, containing the isolated and concentrated exosomes, was added showed significantly higher collagen production compared to the group to which Test Substance 4 was added.

Furthermore, the groups to which Test Substances 3 and 4 were added exhibited significantly higher collagen production compared to the group to which ascorbic acid was added.

Even when the final concentration of Test Substances 1 to 4 was 20%, as shown in FIGS. 32 and 33, the group to which Test Substance 1 (DMEM) was added did not show a significant increase in collagen production compared to the control. In contrast, the groups to which Test Substances 2 to 4 were added exhibited significantly higher collagen production compared to the control. The group to which Test Substance 4, containing the isolated and concentrated proteins, was added showed significantly higher collagen production compared to the group to which Test Substance 2 (culture supernatant) was added. The group to which Test Substance 3, containing the isolated and concentrated exosomes, was added showed significantly higher collagen production compared to the group to which Test Substance 4was added. Furthermore, the groups to which Test Substances 3 and 4 were added exhibited significantly higher collagen production compared to the group to which ascorbic acid was added. Test Substances 3 and 4 also exhibited a concentration-dependent increase in effect.

Moreover, Test Substances 3 and 4 demonstrated greater efficacy even at a final concentration of 1% than Test Substance 2 at a final concentration of 20%.

The results of the present Example indicate that a culture medium used for the maintenance culture of stem cells, isolated and concentrated exosomes derived from stem cells, and isolated and concentrated proteins derived from stem cells promote collagen production by fibroblasts.

### Example 12: FGF-7 Production Assay by Dermal Papilla Cells

A proliferation medium B was prepared by using a dedicated culture medium for dermal papilla cells supplemented with specific additives (fetal bovine serum, insulin-transferrin-triiodothyronine mixture, bovine pituitary extract, and cyproterone acetate) (Cat. No. TMTPGM-250, TOYOBO). Normal human dermal papilla cells (Cat. No. CA60205a, Lot No. 2868, TOYOBO) were suspended in the proliferation medium B at a concentration of 1.2×10⁴ cells/0.3 mL/well, and seeded into type I collagen-coated 48-well plates, followed by incubation in a CO₂ incubator (5% CO₂, 37 °C) for one day.

Each of the Test Substances 1 to 4 was mixed with an additive-free dermal papilla cell culture medium (additive-free assay medium B). In a portion of the wells, the proliferation medium B was replaced with the respective additive-free assay medium B containing each of the test substances. As a control, in another portion of the wells, the proliferation medium B was not replaced with any of the additive-free assay media containing the test substances. As a positive control, minoxidil was mixed with the additive-free assay medium B, and in a portion of the wells, the proliferation medium B was replaced with the minoxidil-containing dermal papilla cell culture medium. The concentration of minoxidil in the minoxidil-containing dermal papilla cell culture medium was 30 μmol/L.

After the medium replacement, the dermal papilla cells were cultured for three days. The culture supernatant was then collected and stored at −80 °C. Subsequently, the supernatant was thawed, and the concentration of fibroblast growth factor 7 (FGF-7) in the culture supernatant was measured using an FGF-7 Human ELISA kit (Cat. No. ab100519, Abcam).

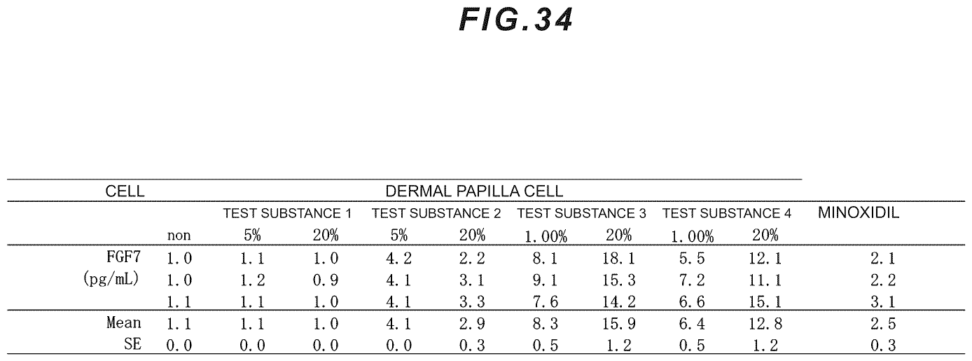

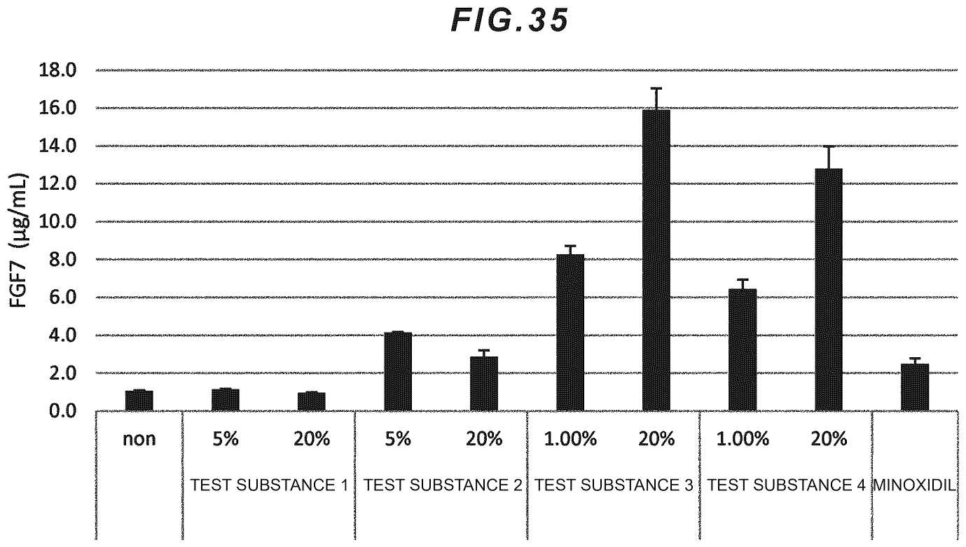

When the final concentrations of Test Substances 1 and 2 were 5%, and the final concentrations of Test Substances 3 and 4 were 1%, as shown in FIGS. 34 and 35, the group to which Test Substance 1, containing DMEM, was added did not show a statistically significant increase in FGF-7 production compared to the control group. In contrast, the groups to which Test Substances 2 through 4 were added exhibited a statistically significant increase in FGF-7 production compared to the control group. The group to which Test Substance 4, containing the isolated and concentrated proteins, was added exhibited a significantly higher FGF-7 production level compared to the group to which Test Substance 2, containing the culture supernatant, was added. The group to which Test Substance 3, containing the isolated and concentrated exosomes, was added exhibited a significantly higher FGF-7 production level compared to the group to which Test Substance 4 was added. Moreover, the groups to which Test Substances 3 and 4 were added showed significantly higher FGF-7 production compared to the group to which minoxidil was added.

Similarly, when the final concentrations of Test Substances 1 to 4 were 20%, as shown in FIGS. 34 and 35, the group to which Test Substance 1, containing DMEM, was added did not exhibit a statistically significant increase in FGF-7 production compared to the control group. In contrast, the groups to which Test Substances 2 through 4 were added exhibited a statistically significant increase in FGF-7 production compared to the control group. The group to which Test Substance 4, containing the isolated and concentrated proteins, was added exhibited a significantly higher FGF-7 production level compared to the group to which Test Substance 2, containing the culture supernatant, was added. The group to which Test Substance 3, containing the isolated and concentrated exosomes, was added exhibited a significantly higher FGF-7 production level compared to the group to which Test Substance 4 was added. Moreover, the groups to which Test Substances 3 and 4 were added showed significantly higher FGF-7 production compared to the group to which minoxidil was added. In addition, Test Substances 3 and 4 exhibited concentration-dependent increase in effect. Furthermore, Test Substances 3 and 4 exhibited higher effects even at a final concentration of 1% than Test Substance 2 at a final concentration of 20%.

The results of the present example demonstrate that the culture medium used for maintenance culture of stem cells, the isolated and concentrated exosomes derived from stem cells, and the isolated and concentrated proteins derived from stem cells promote the production of FGF-7 by dermal papilla cells.

### Example 13: VEGF Production Assay by Dermal Papilla Cells

Dermal papilla cells were cultured in the same manner as in Example 12, the culture supernatants were collected, and stored at −80 °C. Subsequently, the supernatants were thawed, and the concentration of vascular endothelial growth factor (VEGF) in the culture supernatants was measured using the Human VEGF Quantikine ELISA (Cat. No. DVE00, R&D Systems).

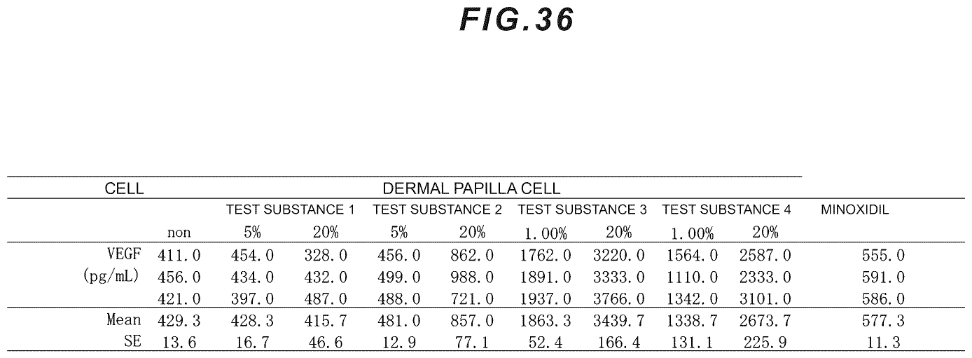

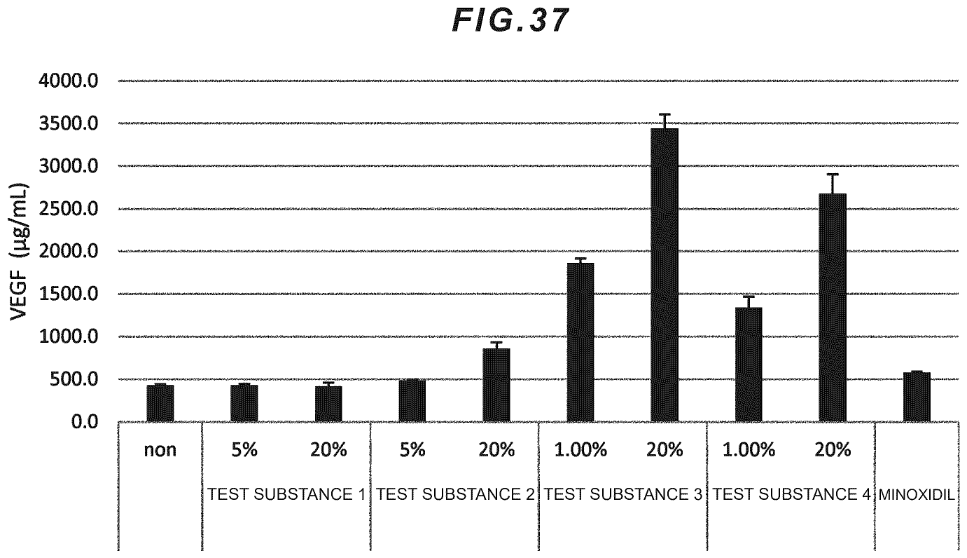

When the final concentrations of Test Substances 1 and 2 were 5%, and the final concentrations of Test Substances 3 and 4 were 1%, as shown in FIGS. 36 and 37, the group to which Test Substance 1, containing DMEM, was added did not exhibit a statistically significant increase in VEGF production compared to the control group. In contrast, the groups to which Test Substances 2 through 4 were added exhibited statistically significant increases in VEGF production compared to the control group. The group to which Test Substance 4, containing the isolated and concentrated proteins, was added exhibited significantly higher VEGF production compared to the group to which Test Substance 2, containing the culture supernatant, was added. The group to which Test Substance 3, containing the isolated and concentrated exosomes, was added exhibited significantly higher VEGF production compared to the group to which Test Substance 4 was added. Furthermore, the groups to which Test Substances 3 and 4 were added showed significantly higher VEGF production compared to the group to which minoxidil was added.

Similarly, when the final concentrations of Test Substances 1 to 4 were 20%, as shown in FIGS. 36 and 37, the group to which Test Substance 1, containing DMEM, was added did not exhibit a statistically significant increase in VEGF production compared to the control group. In contrast, the groups to which Test Substances 2 through 4 were added exhibited statistically significant increases in VEGF production compared to the control group. The group to which Test Substance 4, containing the isolated and concentrated proteins, was added exhibited significantly higher VEGF production compared to the group to which Test Substance 2, containing the culture supernatant, was added. The group to which Test Substance 3, containing the isolated and concentrated exosomes, was added exhibited significantly higher VEGF production compared to the group to which Test Substance 4 was added. Furthermore, the groups to which Test Substances 3 and 4 were added exhibited significantly higher VEGF production compared to the group to which minoxidil was added. Test Substances 3 and 4 exhibited concentration-dependent increase in effect. Moreover, Test Substances 3 and 4 exhibited higher effects even at a final concentration of 1% than did Test Substance 2 at a final concentration of 20%.

The results of the present example demonstrate that the culture medium used for the maintenance culture of stem cells, the isolated and concentrated exosomes derived from stem cells, and the isolated and concentrated proteins derived from stem cells promote the production of VEGF by dermal papilla cells.

### Example 14: Proliferation Assay of Fibroblasts

The same proliferation medium A as used in Example 11 was prepared. Next, normal human dermal fibroblasts derived from an adult donor (KF-4109, Strain No. 01035, Kurabo) were suspended in the proliferation medium A at a concentration of 5×10³ cells/0.1 mL/well, and seeded into a 96-well plate. The cells were then cultured for one day in a CO₂ incubator (5% CO₂, 37 °C).

Subsequently, each of Test Substances 1 through 4 was mixed with the same test medium A as used in Example 11, and the proliferation medium A in some of the wells was replaced with the respective test medium A containing the test substance. As a control, the proliferation medium A in some wells was not replaced with any test medium containing a test substance.

The fibroblasts were cultured for five days in the replaced culture media, and the number of viable cells was measured using the WST-8 assay with the Cell Counting Reagent SF (Cat. No. 07553-15, Nacalai Tesque) and a plate reader (Varioskan MicroPlate Reader, Thermo Scientific).

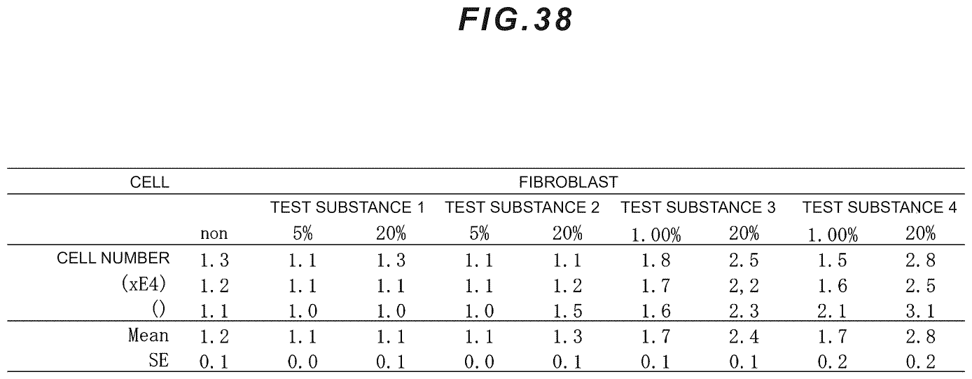

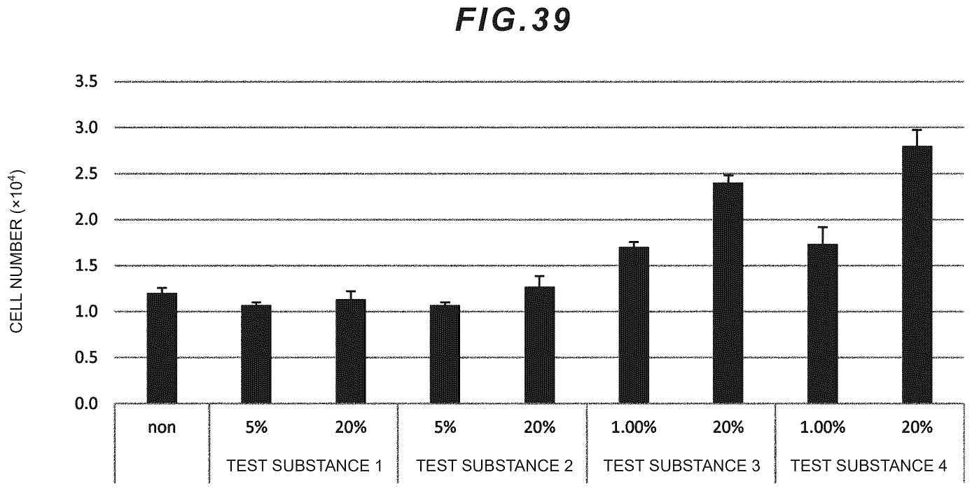

When the final concentrations of Test Substances 1 and 2 were 5%, and the final concentrations of Test Substances 3 and 4 were 1%, as shown in FIGS. 38 and 39, the group to which Test Substance 1, containing DMEM, was added did not show a statistically significant increase in cell number compared to the control. In contrast, the groups to which Test Substances 2 through 4 were added showed a statistically significant increase in cell number compared to the control group without test substance addition. The group to which Test Substance 3, containing the isolated and concentrated exosomes, was added showed a significantly greater number of cells than the group to which Test Substance 2, containing the culture supernatant, was added. Furthermore, the group to which Test Substance 4, containing the isolated and concentrated proteins, was added showed a significantly greater number of cells than the group to which Test Substance 3 was added.

When the final concentrations of Test Substances 1 through 4 were 20%, as shown in FIGS. 38 and 39, the group to which Test Substance 1, containing DMEM, was added did not exhibit a statistically significant increase in cell number compared to the control group. In contrast, the groups to which Test Substances 2 through 4 were added exhibited statistically significant increases in cell number compared to the control group without test substance addition. The group to which Test Substance 3, containing the isolated and concentrated exosomes, was added showed a significantly greater number of cells than the group to which Test Substance 2, containing the culture supernatant, was added. Furthermore, the group to which Test Substance 4, containing the isolated and concentrated proteins, was added showed a significantly greater number of cells than the group to which Test Substance 3 was added. Additionally, Test Substances 3 and 4 exhibited concentration-dependent increase in effect. Moreover, Test Substances 3 and 4, even at a final concentration of 1%, demonstrated greater effects than Test Substance 2 at a final concentration of 20%.

The results of the present example demonstrate that the culture medium used for the maintenance culture of stem cells, the isolated and concentrated exosomes derived from stem cells, and the isolated and concentrated proteins derived from stem cells promote the proliferation of fibroblasts.

### Example 15: Production of Cerebral Neurons

A 12-well plate coated with a solubilized basement membrane preparation (Matrigel, Corning) was prepared, and each well was filled with a feeder-free medium (mTeSR™1, Stemcell Technologies) containing a ROCK (Rho-associated coiled-coil forming kinase) inhibitor (Selleck) at a concentration of 10 nmol/mL.

Induced pluripotent stem (iPS) cells were dissociated using a tissue/cell dissociation solution (Accutase, Innovative Cell Technologies), and seeded into the 12-well plate at a density of 1×10⁵ cells/well. Thereafter, the iPS cells were cultured in the feeder-free medium under gas conditions of 5% carbon dioxide and 20% oxygen for 24 hours.

On the following day, NGN2 (neurogenin 2) RNA linked to an RNA corresponding to a puromycin (Puro) resistance gene was introduced into the iPS cells (Day 0). On Day 2 after the introduction of the NGN2-Puro resistance gene, the culture medium in the wells was replaced with a neural induction medium (N3 medium) containing puromycin at a concentration of 2 μg/mL. The cells were then cultured for three days to selectively eliminate iPS cells that had not received NGN2 and to select for iPS cells into which NGN2 had been introduced. The N3 medium was prepared by adding 10 mL of B27, 5 mL of N2, and 1.6 mL of insulin at a concentration of 6.25 mg/mL to 500 mL of DMEM/F12.

On Day 7 after the introduction of the NGN2-Puro resistance gene, it was confirmed that cerebral neurons positive for vGlu, SATB2, CTIP, and REELIN had been induced from the iPS cells.

### Example 16: Survival Assay of Cerebral Neurons

On Day 7 after the introduction of the NGN2-Puro resistance gene, it was confirmed that cerebral neurons had been induced from the iPS cells. Thereafter, the cerebral neurons were cultured in a neural induction medium supplemented with 10 μM of recombinant amyloid protein and one of Test Substances 2 to 4. The final concentration of each of Test Substances 2 to 4 was 10%. As Comparative Group 1, a group in which neither amyloid β nor any of Test Substances 2 to 4 was added was prepared. As Comparative Group 2, a group in which only amyloid β (10 μM) was added was prepared. The culture medium was replaced once every five days. On Day 17 after the introduction of the NGN2-Puro resistance gene, the number of surviving cerebral neurons per visual field was counted in five fields of view and averaged.

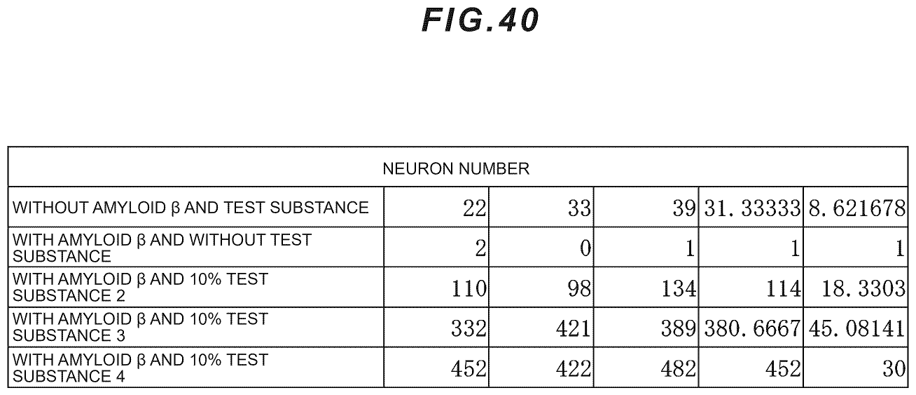

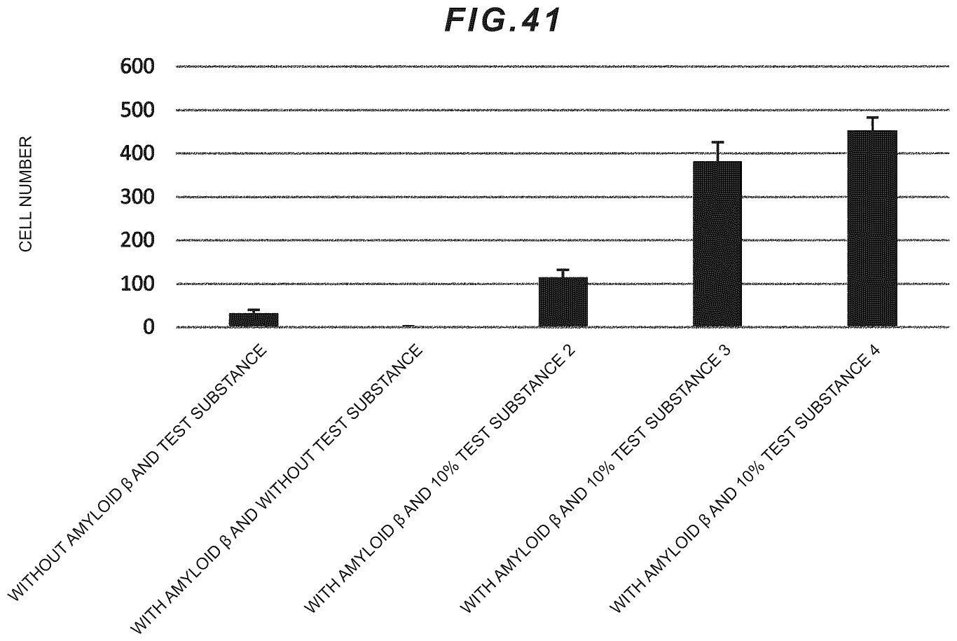

The average number of surviving cerebral neurons per field of view is shown in FIGS. 40 and 41. Compared to Comparative Group 1, Comparative Group 2 exhibited a reduced number of surviving cerebral neurons per field of view. These results indicate that amyloid β induces the death of cerebral neurons. In contrast, the groups to which Test Substances 2 to 4 were added showed a significantly higher number of surviving cells compared to Comparative Group 2. The group to which Test Substance 3, containing the isolated and concentrated exosomes, was added showed a significantly greater number of cells than the group to which Test Substance 2, containing the culture supernatant, was added. The group to which Test Substance 4, containing the isolated and concentrated proteins, was added showed a significantly greater number of cells than the group to which Test Substance 3 was added.

The results of the present example demonstrate that the culture medium used for the maintenance culture of stem cells, the isolated and concentrated exosomes derived from stem cells, and the isolated and concentrated proteins derived from stem cells inhibit the effects of amyloid β and suppress amyloid β-induced death of cerebral neurons.

1.-20. (canceled)

21. A method for inhibiting amyloid β comprising:
administering to a subject a secretion or an extract derived from stem cells or from somatic cells into which reprogramming factors have been introduced.

22. A method for treating or preventing neurological diseases comprising:
administering to a subject a secretion or an extract derived from stem cells or from somatic cells into which reprogramming factors have been introduced.

23. The method of claim 22, wherein the neurological diseases contain dementia.

24. The method of claim 22, wherein the neurological diseases contain Alzheimer's disease.

25. The method of claim 22, wherein the neurological diseases contain Huntington's disease.

26. The method of claim 22, wherein the neurological diseases contain Parkinson's disease.

27. The method of claim 22, wherein the neurological diseases contain amyotrophic lateral sclerosis (ALS).

28. A method for inhibiting neuronal cell death comprising:
administering to a subject a secretion or an extract derived from stem cells or from somatic cells into which reprogramming factors have been introduced.

29. A method for treating or preventing reduction in neuronal cell number comprising:
administering to a subject a secretion or an extract derived from stem cells or from somatic cells into which reprogramming factors have been introduced.
